(19) United States
(12) Patent Application Publication
(10) Pub. No.: US 2015/0110779 A1
(43) Pub. Date: Apr. 23, 2015

(54) METHODS FOR PREDICTING GASTROINTESTINAL IMMUNE-RELATED ADVERSE EVENTS (GI-IRAE) IN PATIENTS TREATED WITH MODULATION OF THE CO-STIMULATORY PATHWAY

(71) Applicant: BRISTOL-MYERS SQUIBB COMPANY, Princeton, NJ (US)

(72) Inventors: Vafa Shahabi, Valley Forge, PA (US); Ruiru Ji, Princeton, NJ (US)

(21) Appl. No.: 14/383,589

(22) PCT Filed: Mar. 15, 2013
(86) PCT No.: PCT/US13/31985

§ 371 Date: Sep. 8, 2014

Related U.S. Application Data
(60) Provisional application No. 61/611,296, filed on Mar. 15, 2012.
(60) Provisional application No. 61/647,003, filed on May 15, 2012.

(51) Int. Cl. C12Q 1/68; A61K 31/58; A61K 31/573; C07K 16/28

(52) U.S. Cl. CPC C12Q 1/6886; C07K 16/2809; A61K 31/58; A61K 31/573; C12Q 2600/112; C12Q 2600/118
USPC 424/133.1; 424/144.1; 506/9; 514/169; 514/174; 514/179

(57) ABSTRACT

The invention described herein relates to diagnostic and therapeutic methods and compositions useful for predicting the likelihood a cancer patient will experience a gastrointestinal immune-related adverse event (GI-irAE) after administration of a pharmaceutically acceptable amount of an activator of the immune system.

This application claims benefit to provisional application U.S. Ser. No. 61/611,296 filed Mar. 15, 2012; and to provisional application U.S. Ser. No. 61/647,003, filed May 15, 2012; under 35 U.S.C. §119(e). The entire teachings of the referenced applications are incorporated herein by reference.

### FIELD OF THE INVENTION

The invention described herein relates to diagnostic and therapeutic methods and compositions useful for predicting the likelihood a cancer patient will experience a gastrointestinal immune-related adverse event (GI-irAE) after administration of a pharmaceutically acceptable amount of an activator of the immune system.

### BACKGROUND OF THE INVENTION

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Anticancer agents have been classified based upon their mechanism of action, and are often referred to as chemotherapeutics. The combination of chemotherapeutics with immune modulating agents has been gaining increasing acceptance in the oncology field.

The vertebrate immune system requires multiple signals to achieve optimal immune activation; see, e.g., Janeway, Cold Spring Harbor Symp. Quant. Biol., 54:1-14 (1989); Paul, W. E., ed., Fundamental Immunology, 4th Edition, Raven Press, N.Y. (1998), particularly Chapters 12 and 13, pp. 411-478. Interactions between T lymphocytes (T cells) and antigen presenting cells (APC's) are essential to the immune response. Levels of many cohesive molecules found on T cells and APC's increase during an immune response (Springer et al., Ann. Rev. Immunol., 5:223-252 (1987); Shaw et al., Curr. Opin. Immunol., Kindt et al., eds., 1:92-97 (1988)); and Hemler, Immunology Today, 9:109-113 (1988)). Increased levels of these molecules may help explain why activated APC's are more effective at stimulating antigen-specific T cell proliferation than are resting APC's (Kaiuchi et al., J. Immunol., 131:109-114 (1983); Kreiger et al., J. Immunol., 135:2937-2945 (1985); McKenzie, J. Immunol., 141:2907-2911 (1988); and Hawrylowicz et al., J. Immunol., 141:4083-4088 (1988)).

T cell immune response is a complex process that involves cell-cell interactions (Springer et al., Ann. Rev. Immunol., 5:223-252 (1987)), particularly between T and accessory cells such as APC's, and production of soluble immune mediators (cytokines or lymphokines) (Dinarello, New Engl. J. Med., 317:940-945 (1987); Sallusto, J. Exp. Med., 179:1109-1118 (1997)). This response is regulated by several T-cell surface receptors, including the T-cell receptor complex (Weiss, Ann. Rev. Immunol., 4:593-619 (1986)) and other “accessory” surface molecules (Allison, Curr. Opin. Immunol., 6:414-419 (1994); Springer, supra (1987)). Many of these accessory molecules are naturally occurring cell surface differentiation (CD) antigens defined by the reactivity of monoclonal antibodies on the surface of cells (McMichael, ed., Leukocyte Typing Iff, Oxford Univ. Press, Oxford, N.Y. (1987)).

Early studies suggested that B lymphocyte activation requires two signals (Bretscher, Science, 169:1042-1049 (1970)) and now it is believed that all lymphocytes require two signals for their optimal activation, an antigen specific or clonal signal, as well as a second, antigen non-specific signal. (Janeway, supra). Freeman (J. Immunol., 143:2714-2722 (1989)) isolated and sequenced a cDNA clone encoding a B cell activation antigen recognized by MAb B7 (Freeman, J. Immunol., 139:3260 (1987)). COS cells transfected with this cDNA have been shown to stain by both labeled MAb B7 and MAb BB-1 (Clark, Human Immunol., 16:100-113 (1986); Yokochi, J. Immunol., 128:823 (1981); Freeman et al., supra (1989); Freeman et al., supra (1987)). In addition, expression of this antigen has been detected on cells of other lineages, such as monocytes (Freeman et al., supra (1989)).

T helper cell (Th) antigenic response requires signals provided by APC's. The first signal is initiated by interaction of the T cell receptor complex (Weiss, J. Clin. Invest., 86:1015 (1990)) with antigen presented in the context of class 1I major histocompatibility complex (MHC) molecules on the APC (Allen, Immunol. Today, 8:270 (1987)). This antigen-specific signal is not sufficient to generate a full response, and in the absence of a second signal may actually lead to clonal inactivation or energy (Schwartz, Science, 248:1349 (1990)). The requirement for a second “costimulatory” signal provided by the MHC has been demonstrated in a number of experimental systems (Schwartz, supra; Weaver et al., Immunol. Today, 11:49 (1990)).

CD28 antigen, a homodimeric glycoprotein of the immunoglobulin superfamily (Aruffo et al., Proc. Natl. Acad. Sci., 84:8573-8577 (1987)), is an accessory molecule found on most mature human T cells (Damle et al., J. Immunol., 131:2296-2300 (1983)). Current evidence suggests that this molecule functions in an alternative T cell activation pathway distinct from that initiated by the T-cell receptor complex (June et al., Mol. Cell. Biol., 7:4472-4481 (1987)). Monoclonal antibodies (MAbs) reactive with CD28 antigen can augment T cell responses initiated by various polyclonal stimuli (reviewed by June et al., supra). These stimulatory effects may result from MAb-induced cytokine production (Thompson et al., Proc. Natl. Acad. Sci., 86:1333-1337 (1989); and Lindsten et al., Science, 244:339-343 (1989)) as a consequence of increased mRNA stabilization (Lindsten et al., supra (1989)). Anti-CD28 mAbs can also have inhibitory effects, i.e., they can block autologous mixed lymphocyte reactions (Damle et al., Proc. Natl. Acad. Sci., 78:5096-6001 (1981)) and activation of antigen-specific T cell clones (Lesslauer et al., Eur. J. Immunol., 16:1289-1296 (1986)).

Some studies have indicated that CD28 is a counter-receptor for the B cell activation antigen, B7/BB-1 (Linsley et al., Proc. Natl. Acad. Sci. USA, 87:5031-5035 (1990)). The B7/BB-1 antigen is hereafter referred to as the “B7 antigen”. The B7 ligands are also members of the immunoglobulin superfamily but have, in contrast to CD28, two Ig domains in their extracellular region, an N-terminal variable (V)-like domain followed by a constant (C)-like domain.

Delivery of a non-specific costimulatory signal to the T cell requires at least two homologous B7 family members found on APC's, B7-1 (also called B7, B7.1, or CD80) and B7-2 (also called B7.2 or CD86), both of which can deliver costimulatory signals to T cells via CD28. Costimulation through CD28 promotes T cell activation.

CD28 has a single extracellular variable region (V)-like domain (Aruffo et al., supra). A homologous molecule, CTLA-4, has been identified by differential screening of a murine cytolytic-T cell cDNA library (Brunet, Nature, 328:267-270 (1987)).

CTLA-4 (CD152) is a T cell surface molecule that was originally identified by differential screening of a murine cytolytic T cell cDNA library (Brunet et al., Nature, 328:267-270 (1987)). CTLA-4 is also a member of the immunoglobulin (Ig) superfamily; CTLA-4 comprises a single extracellular Ig domain. Researchers have reported the cloning and mapping of a gene for the human counterpart of CTLA-4 (Dariavach et al., Eur. J. Immunol., 18:1901-1905 (1988)) to the same chromosomal region (2q33-34) as CD28 (Lafage-Pochitaloff et al., Immunogenetics, 31:198-201 (1990)). Sequence comparison between this human CTLA-4 DNA and that encoding CD28 proteins reveals significant homology of sequence, with the greatest degree of homology in the juxtamembrane and cytoplasmic regions (Brunet et al., supra (1988); Dariavach et al., supra (1988)).

The CTLA-4 is inducibly expressed by T cells. It binds to the B7-family of molecules (primarily CD80 and CD86) on antigen-presenting cells (Chambers et al., Ann. Rev Immunol., 19:565-594 (2001)). When triggered, it inhibits T-cell proliferation and function. Mice genetically deficient in CTLA-4 develop lymphoproliferative disease and autoimmunity (Tivol et al., Immunity., 3:541-547 (1995)). In pre-clinical models, CTLA-4 blockade also augments anti-tumor immunity (Leach et al., Science, 271:1734-1736 (1996); van Elsas et al., J. Exp. Med., 190:355-366 (1999)). These findings led to the development of antibodies that block CTLA-4 for use in cancer immunotherapy.

Blockade of CTLA-4 by a monoclonal antibody leads to the expansion of all T cell populations, with activated CD4⁺ and CD8⁺ T cells mediating tumor cell destruction (Melero et al., Nat. Rev. Cancer, 7:95-106 (2007); Wolchok et al., The Oncologist, 13(Suppl. 4):2-9 (2008)). The antitumor response that results from the administration of anti-CTLA-4 antibodies is believed to be due to an increase in the ratio of effector T cells to regulatory T cells within the tumor microenvironment, rather than simply from changes in T cell populations in the peripheral blood (Quezada et al., J. Clin. Invest., 116:1935-1945 (2006)). One such agent is ipilimumab.

Ipilimumab (previously MDX-010; Medarex Inc., marketed by Bristol-Myers Squibb as YERVOY™) is a fully human anti-human CTLA-4 monoclonal antibody that blocks the binding of CTLA-4 to CD80 and CD86 expressed on antigen presenting cells, thereby, blocking the negative down-regulation of the immune responses elicited by the interaction of these molecules. Initial studies in patients with melanoma showed that ipilimumab could cause objective durable tumor regressions (Phan et al., Proc. Natl. Acad. Sci. USA, 100:8372-8377 (2003)). Also, reductions of serum tumor markers were seen for some patients with ovarian or prostate cancer (Hodi et al., Proc. Natl. Acad. Sci. USA, 100:4712-4717 (2003)). More recently, ipilimumab has demonstrated antitumor activity in patients with advanced melanoma (Weber et al., J. Clin. Oncol., 26:5950-5956 (2008); Weber, Cancer Immunol. Immunother., 58:823-830 (2009)).

Treatment with ipilimumab, however, has been associated with a number of immune-related adverse events (irAEs) such as colitis and skin rash. Predictive biomarkers that can help identify patients who may experience such irAEs could enhance the management of these toxicities. Accordingly, there is a need in the art to identify patients who may have an increased likelihood of experiencing an immune-related adverse event, or who may be predisposed to experiencing an immune-related adverse event, in response to anti-CTLA-4 therapy.

The present inventors have discovered, for the first time, specific biomarkers for predicting after the start of the treatment the likelihood a patient may experience an immune-related adverse event to immunotherapy shortly after the administration of an anti-CTLA-4 therapy.

### SUMMARY OF THE INVENTION

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein an elevated level of CD177 expression relative to said baseline or predetermined level is indicative of an increased likelihood said patient will experience a gastrointestinal immune-related adverse event in response to said therapy.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein an elevated level of CD177 expression relative to said baseline or predetermined level is indicative of an increased likelihood said patient will experience a gastrointestinal immune-related adverse event in response to said therapy, wherein said elevated level of CD177 is measured within about 1, 2, 3, 4, 5, 6, 7, 8, 9, 10, and 11 weeks subsequent to said therapy.

The present invention provides a method for predicting shortly after the start of the treatment the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein an elevated level of CD177 expression relative to said baseline or predetermined level is indicative of an increased likelihood said patient will experience a gastrointestinal immune-related adverse event in response to said therapy, wherein said elevated level of CD177 is measured within about 1, 2, 3, 4, 5, 6, 7, 8, 9, 10, and 11 weeks subsequent to said therapy, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein an elevated level of CD177 expression relative to said baseline or predetermined level is indicative of an increased likelihood said patient will experience a gastrointestinal immune-related adverse event in response to said therapy, wherein said elevated level of CD177 is measured within about 1, 2, 3, 4, 5, 6, 7, 8, 9, 10, 11 or about 12 weeks subsequent to therapy, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein an elevated level of CD177 expression relative to said baseline or predetermined level is indicative of an increased likelihood said patient will experience a gastrointestinal immune-related adverse event in response to said therapy, wherein said elevated level of CD177 is at least about 2 fold, about 3 fold, about 4 fold, about 5 fold, about 6 fold, about 7 fold, about 8 fold, about 9 fold, about 10 fold, at least 11 fold, and at least about 12 fold higher than said baseline or predetermined level.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein an elevated level of CD177 expression relative to said baseline or predetermined level is indicative of an increased likelihood said patient will experience a gastrointestinal immune-related adverse event in response to said therapy, wherein said elevated level of CD177 is measured within about 1, 2, 3, 4, 5, 6, 7, 8, 9, 10, and 11 weeks subsequent to said therapy, wherein said elevated level of CD177 is at least about 2 fold, about 3 fold, about 4 fold, about 5 fold, about 6 fold, about 7 fold, about 8 fold, about 9 fold, about 10 fold, at least 11 fold, and at least about 12 fold higher than said baseline or predetermined level.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein an elevated level of CD177 expression relative to said baseline or predetermined level is indicative of an increased likelihood said patient will experience a gastrointestinal immune-related adverse event in response to said therapy, wherein said elevated level of CD177 is measured within about 1, 2, 3, 4, 5, 6, 7, 8, 9, 10, and 11 weeks subsequent to said therapy, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said elevated level of CD177 is at least about 2 fold, about 3 fold, about 4 fold, about 5 fold, about 6 fold, about 7 fold, about 8 fold, about 9 fold, about 10 fold, at least 11 fold, and at least about 12 fold higher than said baseline or predetermined level.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein an elevated level of CD177 expression relative to said baseline or predetermined level is indicative of an increased likelihood said patient will experience a gastrointestinal immune-related adverse event in response to said therapy, wherein said elevated level of CD177 is measured within about 1, 2, 3, 4, 5, 6, 7, 8, 9, 10, and 11 weeks subsequent to therapy, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab, wherein said elevated level of CD177 is at least about 2 fold, about 3 fold, about 4 fold, about 5 fold, about 6 fold, about 7 fold, about 8 fold, about 9 fold, about 10 fold, at least 11 fold, and at least about 12 fold higher than said baseline or predetermined level.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a predetermined level of CD177 expression, and (iv) continuing to maintain said therapy.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression based upon a patient population that did not experience a gastrointestinal immune-related adverse event, and (iv) continuing to maintain said therapy if said expression level is less than about 4 fold, about 3 fold, and about 2 fold less than said baseline or predetermined level.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, and (iv) continuing to maintain said therapy, and optionally, providing instructions to the treating physician to closely monitor said patient for gastrointestinal immune-related adverse events.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression based upon a patient population that did not experience a gastrointestinal immune-related adverse event, and (iv) continuing to maintain said therapy if said expression level is less than about 4 fold, about 3 fold, and about 2 fold said baseline or predetermined level, and optionally, providing instructions to the treating physician to closely monitor said patient for gastrointestinal immune-related adverse events.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression based upon a patient population that did not experience a gastrointestinal immune-related adverse event, and (iv) continuing to maintain said therapy if said expression level as measured at about week 1, 2, 3, 4, 5, 6, 7, 8, 9, 10, 11, or 12, is less than about 4 fold, about 3 fold, and about 2 fold said baseline or predetermined level, and optionally, providing instructions to the treating physician to closely monitor said patient for gastrointestinal immune-related adverse events.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, and (iv) introducing an alternative treatment regimen for said patient if an elevated level of CD177 expression relative to said baseline or predetermined level is observed.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression based upon a patient population that did not experience a gastrointestinal immune-related adverse event, and (iv) introducing an alternative treatment regimen for said patient if an elevated level of CD177 expression relative to said baseline or predetermined level is observed, wherein said elevated expression is at least about 2 fold, about 3 fold, about 4, or at least about 5 fold more than said baseline or predetermined level.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression based upon a patient population that did not experience a gastrointestinal immune-related adverse event, and (iv) introducing an alternative treatment regimen for said patient if an elevated level of CD177 expression as measured at about week 1, 2, 3, 4, 5, 6, 7, 8, 9, 10, 11, or 12, relative to said baseline or predetermined level is observed, wherein said elevated expression is at least about 2 fold, about 3 fold, about 4, or at least about 5 fold more than said baseline or predetermined level.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, and (iv) introducing an alternative treatment regimen for said patient if an elevated level of CD177 expression relative to said baseline or predetermined level is observed, and optionally, providing instructions to the treating physician to closely monitor said patient for gastrointestinal immune-related adverse events.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, and (iv) introducing an alternative treatment regimen for said patient if an elevated level of CD177 expression relative to said baseline or predetermined level is observed, and optionally, providing instructions to the treating physician to closely monitor said patient for gastrointestinal immune-related adverse events, wherein alternative treatment regimen comprises a regimen selected from the group consisting of: (a) continuing to administer said therapy but altering said regimen by reducing the frequency of said administration; (b) continuing to administer said therapy but altering said regimen by reducing the dose of said therapy; (c) continuing to administer said therapy but altering said regimen by reducing both the dose or frequency of administration of said therapy; (d) continuing to administer said therapy but interrupting the administration of said therapy for a period of time; (e) continuing to administer said therapy but altering said regimen by introducing a steroid into the treatment regimen; (f) continuing to administer said therapy but altering said regimen by introducing infliximab into the treatment regimen; (g) continuing to administer said therapy but altering said regimen by introducing budesonide into the treatment regimen; (h) continuing to administer said therapy but altering said regimen by introducing solumedrol into the treatment regimen; (i) continuing to administer said therapy but altering said regimen by introducing a CD137 agonist into the treatment regimen; and (j) continuing to administer said therapy but altering said regimen by introducing bowel rest and electrolytes into the treatment regimen.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein if said elevated level of CD177 expression has an RMA of at least 8, recommending said therapy be discontinued for a period of time to either prevent or decrease the likelihood said patient will experience a gastrointestinal immune-related adverse event.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein if said elevated level of CD177 expression has an RMA of at least 8, recommending said therapy be discontinued for a period of time to either prevent or decrease the likelihood said patient will experience a gastrointestinal immune-related adverse event.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein if said elevated level of CD177 expression has an RMA of at least 8 at about week 3, 4, 5, 6, 7, 8, 9, 10, 11, or about 12 or longer, recommending said therapy to be discontinued for a period of time to either prevent or decrease the likelihood said patient will experience a gastrointestinal immune-related adverse event.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, and (iv) introducing an alternative treatment regimen for said patient if an elevated level of CD177 expression relative to said baseline or predetermined level is observed, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, and (iv) introducing an alternative treatment regimen for said patient if an elevated level of CD177 expression relative to said baseline or predetermined level is observed, and optionally, providing instructions to the treating physician to closely monitor said patient for gastrointestinal immune-related adverse events, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, and (iv) introducing an alternative treatment regimen for said patient if an elevated level of CD177 expression relative to said baseline or predetermined level is observed, and optionally, providing instructions to the treating physician to closely monitor said patient for gastrointestinal immune-related adverse events, wherein alternative treatment regimen comprises a regimen selected from the group consisting of: (a) continuing to administer said therapy but altering said regimen by reducing the frequency of said administration; (b) continuing to administer said therapy but altering said regimen by reducing the dose of said therapy; (c) continuing to administer said therapy but altering said regimen by reducing both the dose or frequency of administration of said therapy; (d) continuing to administer said therapy but interrupting the administration of said therapy for a period of time; (e) continuing to administer said therapy but altering said regimen by introducing a steroid into the treatment regimen; (f) continuing to administer said therapy but altering said regimen by introducing infliximab into the treatment regimen; (g) continuing to administer said therapy but altering said regimen by introducing budesonide into the treatment regimen; (h) continuing to administer said therapy but altering said regimen by introducing solumedrol into the treatment regimen; (i) continuing to administer said therapy but altering said regimen by introducing a CD137 agonist into the treatment regimen; and (j) continuing to administer said therapy but altering said regimen by introducing bowel rest and electrolytes into the treatment regimen wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein if said elevated level of CD177 expression has an RMA of at least 8, recommending said therapy be discontinued for a period of time to either prevent or decrease the likelihood said patient will experience a gastrointestinal immune-related adverse event wherein said cancer is a solid tumor selected from the group consisting of: melanoma, prostate cancer, lung cancer, non-small cell lung cancer, and small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein if said elevated level of CD177 expression has an RMA of at least 8, recommending said therapy be discontinued for a period of time to either prevent or decrease the likelihood said patient will experience a gastrointestinal immune-related adverse event wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CD177 in a sample from said patient collected subsequent to administration of said therapy; and (iii) comparing said measured expression level to a baseline or predetermined level of CD177 expression, wherein if said elevated level of CD177 expression has an RMA of at least 8 at about week 3, 4, 5, 6, 7, 8, 9, 10, 11, or about 12 or longer, recommending said therapy be discontinued for a period of time to either prevent or decrease the likelihood said patient will experience a gastrointestinal immune-related adverse event wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a kit for use in determining a treatment regimen for an individual with cancer, comprising: (i) a therapeutically effective dose of an anti-CTLA-4 antagonist; (ii) a means for measuring CD177 expression subsequent to the administration of said anti-CTLA-4 antagonist; and (iii) instructions to continue to administer said anti-CTLA-4 antagonist if the level of said CD177 relative to a baseline or predetermined level is less than a threshold level.

The present invention is further directed to a kit for use in determining a treatment regimen for an individual with cancer, comprising: (i) a therapeutically effective dose of an anti-CTLA-4 antagonist; (ii) a means for measuring CD177 expression subsequent to the administration of said anti-CTLA-4 antagonist; and (iii) instructions to continue to administer said anti-CTLA-4 antagonist if the level of said CD177 expression is about 4 fold, 3 fold, or about 2 fold less than a baseline or predetermined level as determined from a patient population that did not experience a gastrointestinal immune-related adverse event.

The present invention is further directed to a kit for use in determining a treatment regimen for an individual with cancer, comprising: (i) a therapeutically effective dose of an anti-CTLA-4 antagonist; (ii) a means for measuring CD177 expression subsequent to the administration of said anti-CTLA-4 antagonist; and (iii) instructions to recommend to discontinue or administer an alternative treatment regimen of said therapy if the level of said CD177 expression is more than about 3 fold, 4 fold, 5 fold, 6 fold, 7 fold, 8 fold, 9 fold, 10 fold, 11 fold, and 12 fold, relative to a baseline or predetermined level.

The present invention is further directed to a kit for use in determining a treatment regimen for an individual with cancer, comprising: (i) a therapeutically effective dose of an anti-CTLA-4 antagonist; (ii) a means for measuring CD177 expression subsequent to the administration of said anti-CTLA-4 antagonist; and (iii) instructions to recommend to discontinue or administer an alternative treatment regimen of said therapy if the level of said CD177 expression is more than about 3 fold, 4 fold, 5 fold, 6 fold, 7 fold, 8 fold, 9 fold, 10 fold, 11 fold, and 12 fold, relative to a baseline or predetermined level as determined from a patient population that did not experience a gastrointestinal immune-related adverse event.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein an elevated level of CEACAM1 expression relative to said baseline or predetermined level is indicative of an increased likelihood said patient will experience a gastrointestinal immune-related adverse event in response to said therapy.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein an elevated level of CEACAM1 expression relative to said baseline or predetermined level is indicative of an increased likelihood said patient will experience a gastrointestinal immune-related adverse event in response to said therapy, wherein said elevated level of CEACAM1 is measured within about 1, 2, 3, 4, 5, 6, 7, 8, 9, 10, and 11 weeks subsequent to said therapy.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein an elevated level of CEACAM1 expression relative to said baseline or predetermined level is indicative of an increased likelihood said patient will experience a gastrointestinal immune-related adverse event in response to said therapy, wherein said elevated level of CEACAM1 is measured within about 1, 2, 3, 4, 5, 6, 7, 8, 9, 10, and 11 weeks subsequent to said therapy, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein an elevated level of CEACAM1 expression relative to said baseline or predetermined level is indicative of an increased likelihood said patient will experience a gastrointestinal immune-related adverse event in response to said therapy, wherein said elevated level of CEACAM1 is measured within about 1, 2, 3, 4, 5, 6, 7, 8, 9, 10, 11 or about 12 weeks subsequent to therapy, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention provides a method for predicting the likelihood a cancer patient will experience a gastrointestinal immune-related adverse event (GI-irAE) shortly after administration of a in response to therapy comprising the administration of a CTLA-4 antagonist, comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein an elevated level of CEACAM1 expression relative to said baseline or predetermined level is indicative of an increased likelihood said patient will experience a gastrointestinal immune-related adverse event in response to said therapy, wherein said elevated level of CEACAM1 is at least about 2 fold, about 3 fold, about 4 fold, about 5 fold, about 6 fold, about 7 fold, about 8 fold, about 9 fold, about 10 fold, at least 11 fold, and at least about 12 fold higher than said baseline or predetermined level.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein an elevated level of CEACAM1 expression relative to said baseline or predetermined level is indicative of an increased likelihood said patient will experience a gastrointestinal immune-related adverse event in response to said therapy, wherein said elevated level of CEACAM1 is measured within about 1, 2, 3, 4, 5, 6, 7, 8, 9, 10, and 11 weeks subsequent to said therapy, wherein said elevated level of CEACAM1 is at least about 2 fold, about 3 fold, about 4 fold, about 5 fold, about 6 fold, about 7 fold, about 8 fold, about 9 fold, about 10 fold, at least 11 fold, and at least about 12 fold higher than said baseline or predetermined level.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein an elevated level of CEACAM1 expression relative to said baseline or predetermined level is indicative of an increased likelihood said patient will experience a gastrointestinal immune-related adverse event in response to said therapy, wherein said elevated level of CEACAM1 is measured within about 1, 2, 3, 4, 5, 6, 7, 8, 9, 10, and 11 weeks subsequent to said therapy, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said elevated level of CEACAM1 is at least about 2 fold, about 3 fold, about 4 fold, about 5 fold, about 6 fold, about 7 fold, about 8 fold, about 9 fold, about 10 fold, at least 11 fold, and at least about 12 fold higher than said baseline or predetermined level.

The present invention provides a method for predicting the likelihood a cancer patient will experience a gastrointestinal immune-related adverse event (GI-irAE) shortly after administration of a therapy comprising the administration of a CTLA-4 antagonist, comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein an elevated level of CEACAM1 expression relative to said baseline or predetermined level is indicative of an increased likelihood said patient will experience a gastrointestinal immune-related adverse event in response to said therapy, wherein said elevated level of CEACAM1 is measured within about 1, 2, 3, 4, 5, 6, 7, 8, 9, 10, and 11 weeks subsequent to therapy, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab, wherein said elevated level of CEACAM1 is at least about 2 fold, about 3 fold, about 4 fold, about 5 fold, about 6 fold, about 7 fold, about 8 fold, about 9 fold, about 10 fold, at least 11 fold, and at least about 12 fold higher than said baseline or predetermined level.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, and (iv) continuing to maintain said therapy.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression based upon a patient population that did not experience a gastrointestinal immune-related adverse event, and (iv) continuing to maintain said therapy if said expression level is about 4 fold, about 3 fold, and about 2 fold less than said baseline or predetermined level.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, and (iv) continuing to maintain said therapy, and optionally, providing instructions to the treating physician to closely monitor said patient for gastrointestinal immune-related adverse events.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression based upon a patient population that did not experience a gastrointestinal immune-related adverse event, and (iv) continuing to maintain said therapy if said expression level is about 4 fold, about 3 fold, and about 2 fold less than said baseline or predetermined level, and optionally, providing instructions to the treating physician to closely monitor said patient for gastrointestinal immune-related adverse events.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression based upon a patient population that did not experience a gastrointestinal immune-related adverse event, and (iv) continuing to maintain said therapy if said expression level as measured at about week 1, 2, 3, 4, 5, 6, 7, 8, 9, 10, 11, or 12, is about 4 fold, about 3 fold, and about 2 fold less than said baseline or predetermined level, and optionally, providing instructions to the treating physician to closely monitor said patient for gastrointestinal immune-related adverse events.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, and (iv) introducing an alternative treatment regimen for said patient if an elevated level of CEACAM1 expression relative to said baseline or predetermined level is observed.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression based upon a patient population that did not experience a gastrointestinal immune-related adverse event, and (iv) introducing an alternative treatment regimen for said patient if an elevated level of CEACAM1 expression relative to said baseline or predetermined level is observed, wherein said elevated expression is at least about 2 fold, about 3 fold, about 4, or at least about 5 fold more than said baseline or predetermined level.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression based upon a patient population that did not experience a gastrointestinal immune-related adverse event, and (iv) introducing an alternative treatment regimen for said patient if an elevated level of CEACAM1 expression as measured at about week 1, 2, 3, 4, 5, 6, 7, 8, 9, 10, 11, or 12, relative to said baseline or predetermined level is observed, wherein said elevated expression is at least about 2 fold, about 3 fold, about 4, or at least about 5 fold more than said baseline or predetermined level.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, and (iv) introducing an alternative treatment regimen for said patient if an elevated level of CEACAM1 expression relative to said baseline or predetermined level is observed, and optionally, providing instructions to the treating physician to closely monitor said patient for gastrointestinal immune-related adverse events.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, and (iv) introducing an alternative treatment regimen for said patient if an elevated level of CEACAM1 expression relative to said baseline or predetermined level is observed, and optionally, providing instructions to the treating physician to closely monitor said patient for gastrointestinal immune-related adverse events, wherein alternative treatment regimen comprises a regimen selected from the group consisting of: (a) continuing to administer said therapy but altering said regimen by reducing the frequency of said administration; (b) continuing to administer said therapy but altering said regimen by reducing the dose of said therapy; (c) continuing to administer said therapy but altering said regimen by reducing both the dose or frequency of administration of said therapy; (d) continuing to administer said therapy but interrupting the administration of said therapy for a period of time; (e) continuing to administer said therapy but altering said regimen by introducing a steroid into the treatment regimen; (f) continuing to administer said therapy but altering said regimen by introducing infliximab into the treatment regimen; (g) continuing to administer said therapy but altering said regimen by introducing budesonide into the treatment regimen; (h) continuing to administer said therapy but altering said regimen by introducing solumedrol into the treatment regimen; (i) continuing to administer said therapy but altering said regimen by introducing a CD137 agonist into the treatment regimen; and (j) continuing to administer said therapy but altering said regimen by introducing bowel rest and electrolytes into the treatment regimen.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein if said elevated level of CEACAM1 has an RMA of at least 8, recommending said therapy be discontinued for a period of time to either prevent or decrease the likelihood said patient will experience a gastrointestinal immune-related adverse event.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein if said elevated level of CEACAM1 expression has an RMA of at least 8, recommending said therapy be discontinued for a period of time to either prevent or decrease the likelihood said patient will experience a gastrointestinal immune-related adverse event.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein if said elevated level of CEACAM1 expression has an RMA of at least 8 at about week 3, 4, 5, 6, 7, 8, 9, 10, 11, or about 12 or longer, recommending said therapy be discontinued for a period of time to either prevent or decrease the likelihood said patient will experience a gastrointestinal immune-related adverse event.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, and (iv) introducing an alternative treatment regimen for said patient if an elevated level of CEACAM1 expression relative to said baseline or predetermined level is observed, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, and (iv) introducing an alternative treatment regimen for said patient if an elevated level of CEACAM1 expression relative to said baseline or predetermined level is observed, and optionally, providing instructions to the treating physician to closely monitor said patient for gastrointestinal immune-related adverse events, wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, and (iv) introducing an alternative treatment regimen for said patient if an elevated level of CEACAM1 expression relative to said baseline or predetermined level is observed, and optionally, providing instructions to the treating physician to closely monitor said patient for gastrointestinal immune-related adverse events, wherein alternative treatment regimen comprises a regimen selected from the group consisting of: (a) continuing to administer said therapy but altering said regimen by reducing the frequency of said administration; (b) continuing to administer said therapy but altering said regimen by reducing the dose of said therapy; (c) continuing to administer said therapy but altering said regimen by reducing both the dose or frequency of administration of said therapy; (d) continuing to administer said therapy but interrupting the administration of said therapy for a period of time; (e) continuing to administer said therapy but altering said regimen by introducing a steroid into the treatment regimen; (f) continuing to administer said therapy but altering said regimen by introducing a infliximab into the treatment regimen; (g) continuing to administer said therapy but altering said regimen by introducing a budesonide into the treatment regimen; (h) continuing to administer said therapy but altering said regimen by introducing a solumedrol into the treatment regimen; (i) continuing to administer said therapy but altering said regimen by introducing a CD137 agonist into the treatment regimen; and (j) continuing to administer said therapy but altering said regimen by introducing bowel rest and electrolytes into the treatment regimen wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein if said elevated level of CEACAM1 expression has an RMA of at least 8, recommending said therapy be discontinued for a period of time to either prevent or decrease the likelihood said patient will experience a gastrointestinal immune-related adverse event wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein if said elevated level of CEACAM1 expression has an RMA of at least 8, recommending said therapy be discontinued for a period of time to either prevent or decrease the likelihood said patient will experience a gastrointestinal immune-related adverse event wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of CEACAM1 in a sample from said patient collected subsequent to administration of said therapy; and (iii) comparing said measured expression level to a baseline or predetermined level of CEACAM1 expression, wherein if said elevated level of CEACAM1 expression has an RMA of at least 8 at about week 3, 4, 5, 6, 7, 8, 9, 10, 11, or about 12 or longer, recommending said therapy be discontinued for a period of time to either prevent or decrease the likelihood said patient will experience a gastrointestinal immune-related adverse event wherein said cancer is a solid tumor selected from the group consisting of: melanoma, prostate cancer, lung cancer, non-small cell lung cancer, small cell lung cancer, gastric cancer, ovarian cancer, brain cancer, and any other cancer disclosed herein, wherein said CTLA-4 antagonist is selected from ipilimumab and tremelimumab.

The present invention is further directed to a kit for use in determining a treatment regimen for an individual with cancer, comprising: (i) a therapeutically effective dose of an anti-CTLA-4 antagonist; (ii) a means for measuring CEACAM1 expression subsequent to the administration of said anti-CTLA-4 antagonist; and (iii) instructions to continue to administer said anti-CTLA-4 antagonist if the level of said CEACAM1 relative to a baseline or predetermined level is less than a threshold level.

The present invention is further directed to a kit for use in determining a treatment regimen for an individual with cancer, comprising: (i) a therapeutically effective dose of an anti-CTLA-4 antagonist; (ii) a means for measuring CEACAM1 expression subsequent to the administration of said anti-CTLA-4 antagonist; and (iii) instructions to continue to administer said anti-CTLA-4 antagonist if the level of said CEACAM1 expression is about 4 fold, 3 fold, or about 2 fold less than a baseline or predetermined level as determined from a patient population that did not experience a gastrointestinal immune-related adverse event.

The present invention is further directed to a kit for use in determining a treatment regimen for an individual with cancer, comprising: (i) a therapeutically effective dose of an anti-CTLA-4 antagonist; (ii) a means for measuring CEACAM1 expression subsequent to the administration of said anti-CTLA-4 antagonist; and (iii) instructions to recommend to discontinue or administer an alternative treatment regimen of said therapy if the level of said CEACAM1 expression is more than about 3 fold, 4 fold, 5 fold, 6 fold, 7 fold, 8 fold, 9 fold, 10 fold, 11 fold, and 12 fold, relative to a baseline or predetermined level.

The present invention is further directed to a kit for use in determining a treatment regimen for an individual with cancer, comprising: (i) a therapeutically effective dose of an anti-CTLA-4 antagonist; (ii) a means for measuring CEACAM1 expression subsequent to the administration of said anti-CTLA-4 antagonist; and (iii) instructions to discontinue or administer an alternative treatment regimen of said therapy if the level of said CEACAM1 expression is more than about 3 fold, 4 fold, 5 fold, 6 fold, 7 fold, 8 fold, 9 fold, 10 fold, 11 fold, and 12 fold, relative to a baseline or predetermined level as determined from a patient population that did not experience a gastrointestinal immune-related adverse event.

The present invention is also directed to methods for predicting shortly after the start of the treatment the likelihood a patient may experience a gastrointestinal immune-related disorder subsequent to the administration of a CTLA-4 antagonist, comprising the steps of: (i) administering to said patient a therapy comprising a CTLA-4 antagonist; (ii) measuring the expression level of at least one biomarker disclosed herein in a sample from said patient collected subsequent to administration of said therapy; and (iii) comparing said measured expression level to a baseline or predetermined level of said biomarker expression, wherein an elevated level of biomarker expression relative to said baseline or predetermined level is indicative of an increased likelihood said patient will experience a gastrointestinal immune-related adverse event in response to said therapy.

The present invention is further directed to a method of treating an individual suffering from cancer with a therapy comprising the administration of a CTLA-4 antagonist comprising the steps of: (i) administering to said patient a therapy comprising a CTLA-4 antagonist; (ii) measuring the expression level of at least one biomarker disclosed herein in a sample from said patient collected subsequent to administration of said therapy; and (iii) comparing said measured expression level to a baseline or predetermined level of said at least one biomarker expression, and (iv) recommending a treatment regimen based upon whether the expression level is above or below said baseline or predetermined level.

The present invention is further directed to a method of treating an individual suffering from cancer with a therapy comprising the administration of ipilimumab, or any other CTLA-4 antagonist, comprising the steps of: (i) administering to said patient a therapy comprising ipilimumab, or any other CTLA-4 antagonist; (ii) measuring the expression level of at least one biomarker selected from CD177 and CEACAM1, in a sample from said patient collected subsequent to administration of said therapy; and (iii) comparing said measured expression level of said at least one biomarker to a baseline or predetermined level of said at least one biomarker, and (iv) recommending said patient cease said therapy if said at least one biomarker has an RMA of at least 8, or is at least about 11 fold higher than said baseline or predetermined level.

The present invention is further directed to a method of treating an individual suffering from cancer with a therapy comprising the administration of ipilimumab, or any other CTLA-4 antagonist, comprising the steps of: (i) administering to said patient a therapy comprising ipilimumab, or any other CTLA-4 antagonist; (ii) measuring the expression level of at least one biomarker selected from CD177 and CEACAM1, in a sample from said patient collected subsequent to administration of said therapy; and (iii) comparing said measured expression level of said at least one biomarker to a baseline or predetermined level of said at least one biomarker, and (iv) recommending said patient be closely monitored and maintain said therapy if said at least one biomarker has an RMA that is less than 8, or is less than about 11 fold higher than said baseline or predetermined level.

The present invention is further directed to a method of treating an individual suffering from cancer with a therapy comprising the administration of ipilimumab, or any other CTLA-4 antagonist, comprising the steps of: (i) administering to said patient a therapy comprising ipilimumab, or any other CTLA-4 antagonist; (ii) measuring the expression level of at least one biomarker selected from CD177 and CEACAM1, in a sample from said patient collected subsequent to administration of said therapy; and (iii) comparing said measured expression level of said at least one biomarker to a baseline or predetermined level of said at least one biomarker, and (iv) recommending said patient be closely monitored and maintain said therapy if said at least one biomarker has an RMA that is less than about 3, or is less than about 1 to 6 fold higher than said baseline or predetermined level.

The present invention is further directed to a kit for use in determining a treatment regimen for an individual with cancer, comprising: (i) a therapeutically effective dose of an anti-CTLA-4 antagonist; (ii) a means for measuring the expression of at least one biomarker disclosed herein subsequent to the administration of said anti-CTLA-4 antagonist; and (iii) instructions to discontinue or administer an alternative treatment regimen of said therapy if the level of said at least biomarker is above or below said baseline or predetermined level.

### BRIEF DESCRIPTION OF THE FIGURES/DRAWINGS

FIG. 1A. Plot of CD177 Expression Versus Weeks Post-Treatment. Log of CD177 expression was plotted over time as a function of weeks post-administration of ipilimumab from patients in studies CA184-004, and -007 (n=162 patients). Whole blood samples were taken at baseline, and 3 and 11 weeks post-treatment. “CA” refers to clinical activity, and “GI-irAE” refers to gastrointestinal immune-related adverse event. As shown, a significant increase in the mean expression of CD177 was observed in patients who experienced serious GI-irAE, independently from their clinical response to ipilimumab. The top seven patients who had the highest expression level of CD177 (above RMA 8) were those who already had or would experience GI-irAE within a few days to three weeks after the 3 week mRNA sample was collected.

FIG. 1B. Plot of Absolute Neurophil Counts and CD177 Expression Versus Weeks Post-Treatment. Absolute neurophil counts and anti-log of CD177 expression was plotted over time as a function of weeks post-administration of ipilimumab from patients in studies CA184-004, and -007 (n=162 patients). Whole blood samples were taken at baseline, and 3 and 11 weeks post-treatment. “GI-irAE” refers to gastrointestinal immune-related adverse event. Numerical values listed beside each “GI-irAE” refer to the grade of the observed gastrointestinal immune-related adverse event. As shown, the mean absolute peripheral blood neutrophil counts (APBNC) increased gradually with ipilimumab treatment regardless of the GI-irAE grades, with the largest increases occurring between weeks 9 and 11. APBNCs also correlated with GI-irAE grades, with patients having larger numbers of APBNCs experiencing more severe GI-irAEs. Importantly, the increase in mean expression of CD177 preceded any significant increase in APBNC, suggesting CD177 expression may be a sensitive, early predictor of GI-irAEs associated with ipilimumab treatment.

FIG. 1C. Consolidated Plot of CD177 Expression Versus Weeks Post-Treatment. A consolidated plot of CD177 expression versus observed gastrointestinal immune-related adverse event grade for all patients in studies CA184-004, and -007 (n=162 patients) according to time in which patient sample was obtained was created. Whole blood samples were taken at baseline, and 3 and 11 weeks post-treatment. “GI-irAE” refers to gastrointestinal immune-related adverse event. Numerical values listed beside each “GI-irAE” refer to the grade of the observed gastrointestinal immune-related adverse event. As shown, CD177 expression at week 3 showed a wide inter-individual variability with considerably higher values in several patients. However, strikingly all seven patients with high levels of CD177 expression (RMA 8) at week 3 reported serious GI-irAEs (G2+) during the course of ipilimumab treatment (see boxed region), suggesting high specificity for this biomarker above this threshold, while patients who experienced GI-irAE with a RMA<8 CD177 expression value at week 3, suggesting decreased sensitivity for this marker below this threshold. While high CD177 expression levels at week 11 post-treatment was observed in both the GI-irAE and No-GI-irAE groups, data from week 11 might be biased because many patients who reported serious adverse events discontinued ipilimumab treatment prior to this time point.

FIG. 1D. Consolidated Plot of CD177 Expression at Week 3 Post-Treatment Versus Serious Gastrointestinal Immune-Related Adverse Event. A consolidated plot of CD177 expression versus observed gastrointestinal immune-related adverse event grade for all patients in studies CA184-004, and -007 (n=162 patients) at 3 weeks post-ipilimumab administration. Whole blood samples were taken at week 3 post-treatment. “GI-irAE” refers to gastrointestinal immune-related adverse event. Numerical values listed within each circle refer to the worst grade of the observed gastrointestinal immune-related adverse event. As shown, Out of 44 reported cases of serious GI-irAE (G2+) in CA184004 and 007 studies, with matching mRNA data at week 3, six patients reported the first serious incident before or on day 21±3 days (the nominal date when the mRNA sample was collected), whereas the other 38 cases were reported after this date. The four highest values of the CD177 expression belonged to patients who reported the first serious GI-irAE between days 26 (2-5 days after mRNA collection) and 43 after receiving the first dose of ipilimumab. One of the patients who showed high CD177 expression levels at week 3, reported a G2 GI-irAE on day 72 (marked in gray shade circle), but progressed to a Grade 4 which ultimately led to a fatal GI perforation in this patient. These data suggest that although considerable increases in CD177 gene expression within days to several weeks after post-ipilimumab administration is closely associated with the onset of GI-irAE, early increases in CD177 expression may also serve to predict GI-irAEs that develop at much later time points.

FIG. 2A. Plot of Expression Observed for Neutrophil Associated and Non-Neutrophil Associated Genes Relative to CD177 Expression. A plot of mean expression observed for neutrophil associated and non-neutrophil associated genes relative to CD177 mean expression observed for all patients in studies CA184-004, and -007 (n=162 patients) at 3 weeks post-ipilimumab administration. Whole blood samples were taken at week 3 post-treatment. As shown, expression of the neutrophil associated genes was found to be greater in mRNA samples that had high levels of CD177 expression.

FIG. 2B. Plot of CEACAM1 Expression Versus Weeks Post-Treatment. Log of CEACAM1 expression was plotted over time as a function of weeks post-administration of ipilimumab from patients in studies CA184-004, and -007 (n=162 patients). Whole blood samples were taken at baseline, and 3 and 11 weeks post-treatment. “GI-irAE” refers to gastrointestinal immune-related adverse event. As shown, a significant increase in the mean expression of CEACAM1 was observed in patients who experienced serious GI-irAE. Thus, the mean gene expression of CEACAM1, showed the exact same pattern as CD177.

FIG. 2C. Heatmap of Neutrophil Associated Genes Versus Serious Gastrointestinal Immune-Related Adverse Event. A heatmap showing the relative expression pattern of neutrophil associated genes as a function of incidence of gastrointestinal immune-related adverse events was created based upon expression observed three weeks post-administration of ipilimumab from patients in studies CA184-004, and -007 (n=162 patients). Whole blood samples were taken at 3 weeks post-treatment. “GI-irAE” refers to gastrointestinal immune-related adverse event. As shown, expression patterns of neutrophil associated genes (other than for CD177 and CEACAM1) however did not show significant associations with the occurrence of GI-irAEs.

FIG. 3. Plot of Immunoglobulin-Related Gene Expression Versus Weeks Post-Treatment. Fold change of immunoglobulin-related gene expression was plotted over time as a function of weeks post-administration of ipilimumab from patients in studies CA184-004, and -007 (n=162 patients), and grouped according to patients experiencing at least a grade 2 gastrointestinal immune-related adverse event. Whole blood samples were taken at baseline, and 3 and 11 weeks post-treatment. “GI-irAE” refers to gastrointestinal immune-related adverse event. As shown, significant increases in expression of immunoglobulin-related genes at week 11 post-treatment was observed. A slight increase in expression of some of these genes was also apparent in post-treatment samples at 3 weeks post-ipilimumab administration suggesting an early onset of the antibody response with much larger expansion at later time points for those patients experiencing a grade 2 or higher gastrointestinal immune-related adverse event, and a comparatively lower level of expansion of immunoglobulin-related gene expression for those patients that did not experience a grade 2 or higher gastrointestinal immune-related adverse event.

FIG. 4. ROC Curve of CD177 Expression at Week 3 as a Predictor of GI-irAE. The plot included 155 patients with known GI-irAE status and CD177 expression values.

### DETAILED DESCRIPTION OF THE INVENTION

The present invention is based on data from two phase II clinical trials that demonstrated patients who exhibited elevated expression levels of CD177 and/or CEACAM1 after the administration of the anti-CTLA-4 antibody, ipilimumab, had an increased likelihood of developing a gastrointestinal immune-related adverse event (GI-irAE) later in therapy. The likelihood a patient may experience a GI-irAE was proportional to the level of expression of CD177 and/or CEACAM1 at 3 weeks and 11 weeks relative to the level of expression of these genes that was observed in patients that did not experience a GI-irAE. This correlation was independent from the patient's clinical response to ipilimumab.

Accordingly, CD177 and/or CEACAM1 expression levels are positively associated with, and are thus useful as a predictive indicator for, identifying patients that may have an increased likelihood of developing a GI-irAE soon after being administered an anti-CTLA-4 modulatory or co-stimulatory pathway modulator, such as, for example, ipilimumab.

The present invention also discloses a correlation between elevated expression levels of Ig-associated genes over time with the incidence of GI-irAE.

For the purposes of the present invention, the phrase “positively associated” refers to a general condition where a higher CD177 and/or CEACAM1 expression value for a given patient relative to a baseline or predetermined level suggests that the patient may have a correspondingly higher likelihood of developing a GI-irAE.

In addition, elevated CD177 and/or CEACAM1 expression levels at specific time points after administration of an anti-CTLA-4 modulator are useful as a predictive indicator for identifying patients who may have a higher likelihood of developing a GI-irAE later during therapy. Specifically, patients that presented with elevated CD177 and/or CEACAM1 expression levels 3 weeks post administration of a co-stimulatory pathway modulator, such as for example, ipilimumab, resulted in significantly increased likelihood the patient may develop a GI-irAE. Furthermore, the magnitude of CD177 and/or CEACAM1 expression levels 3 weeks post-administration of an anti-CTLA-4 modulator relative to a baseline or predetermined level was positively associated with likelihood a patient may develop a severe GI-irAE, with higher levels of expression being associated with an increased likelihood of developing a more severe GI-irAE grade. Specifically, there was a strong likelihood a patient that presented with CD177 and/or CEACAM1 expression that was greater than or equal to RMA 8 on a log scale relative to the expression level of CD177 and/or CEACAM1 in patients that did not experience a GI-irAE would later develop an GI-irAE later in therapy.

In addition, elevated CD177 and/or CEACAM1 expression may be useful for identifying patients who may require prophylactic treatment to prevent or decrease the likelihood that a patient may develop the incidence of a GI-irAE.

The use of CD177 and/or CEACAM1 expression as a diagnostic is also useful for, among other things, assisting health care professionals in developing tailored treatment regimens suitable for the condition(s) presented herein, particularly for the treatment of melanoma.

The teachings of the present invention are believed to be the first association between high expression levels of CD177 and/or CEACAM1 and the likelihood a patient may develop a GI-irAE subsequent to the administration of a co-stimulatory pathway modulator, such as ipilimumab.

For the purposes of the present invention, the expression level of CD177 and/or CEACAM1 may be measured during a period of time shortly after administration of an anti-CTLA-4 antagonist (“early post-administration period”). A patient's base expression level of CD177 and/or CEACAM1 may optionally be measured prior to the first therapeutic dose as well as a baseline.

In one embodiment of the present invention, the early post-administration period during which a patient's level of CD177 and/or CEACAM1 expression is measured may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours post-administration of an anti-CTLA-4 antagonist.

In another embodiment, the early post-administration period during which a patient's level of CD177 and/or CEACAM1 expression is measured may be about 1, about 2, about 3, about 4, about 5, about 6, or about 7 days post-administration of an anti-CTLA-4 antagonist. In this context, the term “about” shall be construed to mean±1, 2, 3, 4, 5, 6, 7, 8, 9, 10, 11, or 12 hours more or less than the stated period.

In another embodiment, the early post-administration period during which a patient's level of CD177 and/or CEACAM1 expression is measured may be about 1, about 2, about 3, about 4, about 5, about 6, about 7, about 8, about 9, about 10, about 11, or about 12 weeks post-administration of an anti-CTLA-4 antagonist. In this context, the term “about” shall be construed to mean±1, 2, 3, 4, 5, 6, or 7 days more or less than the stated period.

For the purposes of the present invention, the expression level of CD177 and/or CEACAM1 may also be measured during a period of time after administration of an anti-CTLA-4 antagonist (“late post-administration period”). A patient's base expression level of CD177 and/or CEACAM1 may optionally be measured prior to the first therapeutic dose as well as a baseline.

In one embodiment of the present invention, the late post-administration period during which a patient's level of CD177 and/or CEACAM1 expression is measured may be about 4, about 5, about 6, about 7, about 8, about 9, about 10, about 11, about 12, about 13, about 14, about 15, about 16, about 17, about 18, about 19, and about 20 weeks post-administration of an anti-CTLA-4 antagonist. In this context, the term “about” shall be construed to mean±1, 2, 3, 4, 5, 6, or 7 days more or less than the stated period.

In one embodiment, the level of expression of CD177 and/or CEACAM1 of a patient sample during the early post-administration period is compared to a baseline or predetermined level of CD177 and/or CEACAM1 expression. Such a baseline or predetermined level may be a value predetermined by measuring the patient's own base level of expression prior to the administration of an anti-CTLA-4 antagonist. Alternatively, a baseline or predetermined level may be a value predetermined by calculating the average value observed in patients prior to the administration of an anti-CTLA-4 antagonist. Alternatively, the baseline or predetermined level may be a value predetermined by calculating the average value observed in patients that did not experience a gastrointestinal immune-related adverse event prior to the administration of an anti-CTLA-4 antagonist. Alternatively, the baseline or predetermined level may be a value predetermined by calculating the average value observed in patients that did not experience a gastrointestinal immune-related adverse event subsequent to the administration of an anti-CTLA-4 antagonist. Alternatively, the baseline or predetermined level may be a value predetermined by calculating the average value observed in patients that did not experience a gastrointestinal immune-related adverse event subsequent to the administration of an anti-CTLA-4 antagonist as measured at the same or similar period of time that the patient sample to be tested was measured.

In one embodiment, the level of expression of CD177 and/or CEACAM1 of a patient sample during the late post-administration period is compared to a baseline or predetermined level of CD177 and/or CEACAM1 expression. Such a baseline or predetermined level may be a value determined by measuring the patient's own base level of expression prior to the administration of an anti-CTLA-4 antagonist. Alternatively, a baseline or predetermined level may be a value determined by calculating the average value observed in patients prior to the administration of an anti-CTLA-4 antagonist. Alternatively, the baseline or predetermined level may be a value determined by calculating the average value observed in patients that did not experience a gastrointestinal immune-related adverse event prior to the administration of an anti-CTLA-4 antagonist. Alternatively, the baseline or predetermined level may be a value determined by calculating the average value observed in patients that did not experience a gastrointestinal immune-related adverse event subsequent to the administration of an anti-CTLA-4 antagonist. Alternatively, the baseline or predetermined level may be a value determined by calculating the average value observed in patients that did not experience a gastrointestinal immune-related adverse event subsequent to the administration of an anti-CTLA-4 antagonist as measured at the same or similar period of time that the patient sample to be tested was measured. The baseline or predetermined value may be adjusted by normalizing the measured value using one or more housekeeping genes.

In one embodiment, the determination of whether a patient's CD177 and/or CEACAM1 expression is elevated relative to a baseline or predetermined level may be assessed by comparing the two values. For the purposes of the present invention, if the measured value is greater than the baseline or predetermined value, the patient is considered to have an elevated level of expression of CD177 and/or CEACAM1. Likewise, if a measured expression level is less than or equal to the baseline or predetermined value, then said patient is considered to have a decreased level of expression of CD177 and/or CEACAM1. In one embodiment of the present invention, a patient is considered to have an elevated expression level of CD177 and/or CEACAM1 if the measured level of expression is at least about 1.5-fold or greater than the baseline or predetermined level. In one embodiment of the present invention, a patient is considered to have an elevated expression level of CD177 and/or CEACAM1 if the measured level of expression is about two-fold or greater than the baseline or predetermined level. In one embodiment of the present invention, a patient is considered to have an elevated expression level of CD177 and/or CEACAM1 if the measured level of expression is about three-fold or greater than the baseline or predetermined level. In one embodiment of the present invention, a patient is considered to have an elevated expression level of CD177 and/or CEACAM1 if the measured level of expression is about four-fold or greater than the baseline or predetermined level. In one embodiment of the present invention, a patient is considered to have an elevated expression level of CD177 and/or CEACAM1 if the measured level of expression is about five-fold or greater than the baseline or predetermined level. In one embodiment of the present invention, a patient is considered to have an elevated expression level of CD177 and/or CEACAM1 if the measured level of expression is about six-fold or greater than the baseline or predetermined level. In one embodiment of the present invention, a patient is considered to have an elevated expression level of CD177 and/or CEACAM1 if the measured level of expression is about seven-fold or greater than the baseline or predetermined level. In one embodiment of the present invention, a patient is considered to have an elevated expression level of CD177 and/or CEACAM1 if the measured level of expression is about eight-fold or greater than the baseline or predetermined level. In one embodiment of the present invention, a patient is considered to have an elevated expression level of CD177 and/or CEACAM1 if the measured level of expression is about nine-fold or greater than the baseline or predetermined level. In one embodiment of the present invention, a patient is considered to have an elevated expression level of CD177 and/or CEACAM1 if the measured level of expression is about ten-fold or greater than the baseline or predetermined level. In one embodiment of the present invention, a patient is considered to have an elevated expression level of CD177 and/or CEACAM1 if the measured level of expression is about eleven-fold or greater than the baseline or predetermined level. In one embodiment of the present invention, a patient is considered to have an elevated expression level of CD177 and/or CEACAM1 if the measured level of expression is about twelve-fold or greater than the baseline or predetermined level. In one embodiment of the present invention, a patient is considered to have an elevated expression level of CD177 and/or CEACAM1 if the measured level of expression is about thirteen-fold or greater than the baseline or predetermined level. In one embodiment of the present invention, a patient is considered to have an elevated expression level of CD177 and/or CEACAM1 if the measured level of expression is about fourteen-fold or greater than the baseline or predetermined level, or even about fifteen, sixteen, seventeen, eighteen, nineteen, 20, 21, 22 or about 25 fold greater. In this context, the term “about” should be construed to mean about ±0.1, ±0.2, ±0.3, ±0.4, ±0.5, ±0.6, ±0.7, ±0.8, ±0.9, above or below the baseline or predetermined value.

The present invention contemplates that any given patient response to a therapy is complex, and likely depends upon a number of factors, including, but not limited to a patient's genetic background, diet, lifestyle, or may even depend upon the presence or absence of confounding patient conditions such as the presence of other disorders at the time the therapy is administered, or that may arise during the course of therapeutic administration, etc. Such factors may obscure or delay the presentation of a true, elevated level of expression of CD177 and/or CEACAM1, such that the presence of such factors may cause the value of said expression to be slightly less than or equal to the fold change intervals outlined herein. Accordingly, for the purposes of the present invention, the definition of elevated level of expression of CD177 and/or CEACAM1 may also include levels of expression that that are slightly less than about ±10%, about ±5%, or even about ±1% of being about at the fold threshold.

The transformation of a patient's measured level of CD177 and/or CEACAM1 post administration of an anti-CTLA-4 modulator into a probability for predicting patient developing a GI-irAE may depend upon a number of factors, including, but not limited to the patient's health, the condition for which the patient is being treated, the therapy the patient has been administered, the dose of the therapy administered, the frequency of the dosing regimen, or any other considerations a health care professional may take into account. Nonetheless, a elevated CD177 and/or CEACAM1 expression levels post administration of an anti-CTLA-4 modulator may be transformed into a probability that predicts a patient will have an increased probability of developing a GI-irAE with higher levels of expression correlating with higher GI-irAE probability; while a similar, non-elevated, or decreased CD177 and/or CEACAM1 expression levels post administration of an anti-CTLA-4 modulator may be transformed into a probability that predicts a patient will have a decreased probability of developing a GI-irAE.

The present invention contemplates that the present invention may be carried out in a number of different modes. For example, in one mode, the present invention contemplates at least one or more of the steps of the diagnostic method being performed by a computer. For example, the measurement of a patient's CD177 and/or CEACAM1 expression level may be performed by a computer. In addition, the measurement of a patient's CD177 and/or CEACAM1 expression level either alone or in conjunction with comparing said level to a baseline or predetermined level may be performed by a computer. In addition, the determination of whether a patient's CD177 and/or CEACAM1 expression level is elevated, the same, or less than a baseline or predetermined value, may be performed by a computer. In addition, the transformation of a patient's CD177 and/or CEACAM1 expression level into the probability of a patient developing a GI-irAE may be performed by a computer. One of skill in the computer programming arts could readily draft software that performs the steps of the invention algorithmically.

The computer for carrying out one mode of the present invention may comprise a CPU, ROM, standard I/O for receiving and outputting instructions and responses, algorithms for carrying out specific steps of the present invention, operating system software, and the like. The computer also may include a display means for conveying I/O information to the user (e.g., monitor, LCD, CRT, etc.), and may also include an entry means (e.g., keyboard, mouse, trackball, touch pad, etc.) to permit user interaction.

The phrase “gastrointestinal immune-related adverse event” or “GI-irAE” refers to adverse events that result either directly or indirectly from the administration of an immune system modulator. Within the context of administering an anti-CTLA-4 modulator, GI-irAE encompassed adverse events including colitis, skin rash, diarrhea, and other conditions described herein or otherwise known in the art. GI-irAE are graded from Grade 1 to Grade 5 according to severity with Grade 1 corresponding to mild adverse event, such as rash or diarrhea, Grade 2 corresponding to moderate adverse events such as prolonged diarrhea, Grade 3 corresponding to severe adverse events such as colitis, and Grade 4 corresponding to life threatening or disabling adverse events such as bowel perforation, and Grade 5 corresponding to death related to an adverse event.

The phrase “anti-CTLA-4 modulator” or “co-stimulatory pathway modulator”, generally refers to an immunostimulant or T-cell activator, and also encompasses any agent that is capable of disrupting the ability of CD28 antigen to bind to its cognate ligand, to inhibit the ability of CTLA-4 to bind to its cognate ligand, to augment T cell responses via the co-stimulatory pathway, to disrupt the ability of B7 to bind to CD28 and/or CTLA-4, to disrupt the ability of B7 to activate the co-stimulatory pathway, to disrupt the ability of CD80 to bind to CD28 and/or CTLA-4, to disrupt the ability of CD80 to activate the co-stimulatory pathway, to disrupt the ability of CD86 to bind to CD28 and/or CTLA-4, to disrupt the ability of CD86 to activate the co-stimulatory pathway, and to disrupt the co-stimulatory pathway, in general from being activated. This necessarily includes small molecule inhibitors of CD28, CD80, CD86, CTLA-4, among other members of the co-stimulatory pathway; antibodies directed to CD28, CD80, CD86, CTLA-4, among other members of the co-stimulatory pathway; antisense molecules directed against CD28, CD80, CD86, CTLA-4, among other members of the co-stimulatory pathway; ADNECTINS™ directed against CD28, CD80, CD86, CTLA-4, among other members of the co-stimulatory pathway, RNAi inhibitors (both single and double stranded) of CD28, CD80, CD86, CTLA-4, among other members of the co-stimulatory pathway, among other anti-CTLA-4 antagonists.

Suitable anti-CTLA-4 antagonist agents for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab, anti-CD28 antibodies, anti-CTLA-4 ADNECTINS™, anti-CTLA-4 domain antibodies, single chain anti-CTLA-4 fragments, heavy chain anti-CTLA-4 fragments, light chain anti-CTLA-4 fragments, modulators of the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145), Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. Each of these references is specifically incorporated herein by reference for purposes of description of CTLA-4 antibodies. A preferred clinical CTLA-4 antibody is human monoclonal antibody 10D1 (also referred to as MDX-010 and ipilimumab and available from Medarex, Inc., Bloomsbury, N.J.), disclosed in WO 01/14424.

As is known in the art, ipilimumab refers to an anti-CTLA-4 antibody, and is a fully human IgG₁ antibody derived from transgenic mice having human genes encoding heavy and light chains to generate a functional human repertoire. Ipilimumab can also be referred to by its CAS Registry No. 477202-00-9, and is disclosed as antibody 10DI in PCT Publication No. WO 01/14424, incorporated herein by reference in its entirety and for all purposes. Specifically, ipilimumab describes a human monoclonal antibody or antigen-binding portion thereof that specifically binds to CTLA-4, comprising a light chain variable region and a heavy chain variable region having a light chain variable region comprised of SEQ ID NO:5, and comprising a heavy chain region comprised of SEQ ID NO:6. Pharmaceutical compositions of ipilimumab include all pharmaceutically acceptable compositions comprising ipilimumab and one or more diluents, vehicles and/or excipients. Examples of a pharmaceutical composition comprising ipilimumab are provided in PCT Publication No. WO 2007/67959. Ipilimumab may be administered by I.V.

Light Chain Variable Region for Ipilimumab:
(SEQ ID NO: 1)
EIVLTQSPGTLSLSPGERATLSCRASQSVGSSYLAWYQQKPGQAPRLLI
YGAFSRATGIPDRFSGSGSGTDFTLTISRLEPEDFAVYYCQQYGSSPWT
FGQGTKVEIK
Heavy Chain Variable Region for Ipilimumab:
(SEQ ID NO: 2)
QVQLVESGGGVVQPGRSLRLSCAASGFTFSSYTMHWVRQAPGKGLEWVT
FISYDGNNKYYADSVKGRFTISRDNSKNTLYLQMNSLRAEDTAIYYCAR
TGWLGPFDYWGQGTLVTVSS

As noted elsewhere herein, the expression level of CD177, CEACAM1, and/or any other marker disclosed herein may be useful for predicting whether a patient may develop a GI-irAE subsequent to the administration of one or more anti-CTLA-4 antagonists, either alone or in combination with a peptide antigen (e.g., gp100), in addition to or in conjunction with an anti-proliferative agent disclosed herein. A non-limiting example of a peptide antigen would be a gp100 peptide comprising, or alternatively consisting of, the sequence selected from the group consisting of: IMDQVPFSV (SEQ ID NO:3), and YLEPGPVTV (SEQ ID NO:4). Such a peptide may be administered orally, or preferably at 1 mg emulsified in incomplete Freund's adjuvant (IFA) injected s.c. in one extremity, and 1 mg of either the same or a different peptide emulsified in IFA may be injected in another extremity.

Disorders for which the present invention may be useful for predicting whether a patient may develop a GI-irAE subsequent to the administration of one or more immunotherapy and/or co-stimulatory pathway modulation, for example, through the administration of ipilimumab, include, but are not limited to melanoma, primary melanoma, unresectable stage III or IV malignant melanoma, lung cancer, non-small cell lung cancer, small cell lung cancer, and prostate cancer.

Additional disorders for which the present invention may be useful for predicting whether a patient may develop a GI-irAE subsequent to the administration of one or more immunotherapy and/or co-stimulatory pathway modulation, for example, through the administration of ipilimumab, include, but are not limited to glioma, gastrointestinal cancer, renal cancer, ovarian cancer, liver cancer, colorectal cancer, endometrial cancer, kidney cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

The terms “treating”, “treatment” and “therapy” as used herein refer to curative therapy, prophylactic therapy, preventative therapy, and mitigating disease therapy.

The phrase “more aggressive dosing regimen” or “increased dosing frequency regimen”, as used herein refers to a dosing regimen that necessarily exceeds the basal and/or prescribed dosing regimen of a co-stimulatory pathway modulator, preferably ipilimumab, either due to an increased dosing frequency (about once a week, about bi-weekly, about once daily, about twice daily, etc.), increased or escalated dose (about 11, about 12, about 13, about 14, about 15, about 16, about 17, about 18, about 19, about 20, about 21, about 22, about 23, about 24, about 25, about 26, about 27, about 28, about 29, about 30, about 35, about 40 mg/ml), or the route of administration which may result in an increased, bio-available level of said co-stimulatory modulator.

It is to be understood this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, preferably ±5%, or ±1%, or as little as ±0.1% from the specified value, as such variations are appropriate to perform the disclosed methods.

Treatment regimens can be established based upon determining whether a patient exhibits an elevated expression level of CD177, CEACAM1, and/or other biomarker disclosed herein, subsequent to the administration of a co-stimulatory pathway modulator, such as ipilimumab, or other therapy described herein, such as chemotherapy. If an elevated expression level of CD177, CEACAM1, and/or other biomarker disclosed herein is detected in the sample from said patient subsequent to administration of a co-stimulatory pathway modulator, a treatment regimen can be developed appropriately. For example, if a significantly elevated expression level of CD177, CEACAM1, and/or other biomarker disclosed herein is detected, it may indicate said patient has an increased likelihood of developing a GI-irAE to said co-stimulatory pathway modulator therapy, and thus the treating physician may be advised to closely monitor said patient for early symptoms of an immune related adverse reaction. If immune related adverse reaction symptoms appear, an intervention treatment regiment may be warranted. Alternatively, if only a modestly elevated expression level of CD177, CEACAM1, and/or other biomarker disclosed herein is detected, it may also indicate said patient has a decreased likelihood of developing a GI-irAE to said co-stimulatory pathway modulator therapy, and thus the recommended treatment regiment should be maintained.

In one aspect, an intervention treatment regiment may include recommending that the treating physician administer corticosteroids either before, after or concurrent with said co-stimulatory pathway modulator.

In one aspect, an intervention treatment regiment may include recommending bowel rest for the patient either before, after or concurrent with said co-stimulatory pathway modulator. Such an intervention treatment regimen may be recommended either alone or in conjunction with corticosteroids or other treatment outlined herein.

In one aspect, an intervention treatment regiment may include recommending the administration of LOMOTIL® and/or IMODIUM® to the patient either before, after or concurrent with said co-stimulatory pathway modulator. Such an intervention treatment regimen may be recommended either alone or in conjunction with corticosteroids or other treatment outlined herein.

In another aspect, another intervention treatment regiment may include recommending a decreased dosing level of a co-stimulatory pathway modulator, such as ipilimumab, to be about 10, 20, 30, 40, 50, 60, 70, 80, 90, or 95% less than the typical co-stimulatory pathway modulator dose for a particular indication or individual (e.g., about 3 mg/kg, about 2.5 mg/kg, about 2 mg/kg, about 1.5 mg/kg, about 1 mg/kg, about 0.9 mg/kg, about 0.8 mg/kg, about 0.7 mg/kg, about 0.6 mg/kg, about 0.5 mg/kg, about 0.4 mg/kg, about 0.3 mg/kg, about 0.2 mg/kg, about 0.1 mg/kg, about 0.05 mg/kg, about 0.01 mg/kg, about 0.005 mg/kg), or about 1.5×, 2×, 2.5×, 3×, 3.5×, 4×, 4.5×, 5×, 6×, 7×, 8×, 9×, or 10× less co-stimulatory pathway modulator than the typical co-stimulatory pathway modulator dose for a particular indication or for individual. Such an intervention treatment regimen may be recommended either alone or in conjunction with corticosteroids or other treatment outlined herein.

In another aspect, an intervention treatment regiment may include recommending a reduced frequency of administration of a co-stimulatory pathway modulator, such as ipilimumab. For example, such a reduced frequency of administration of a co-stimulatory pathway modulator may include an administration as follows: about once every 3 weeks, about once every 3.5 weeks, about once every 4 weeks, about once every 4.5 weeks, about once every 5 weeks, about once every 5.5 weeks, about once every 6 weeks, about once every 6.5 weeks, about once every 7 weeks, about once every 7.5 weeks, about once every 8 weeks, about once every 8.5 weeks, about once every 9 weeks, about once every 2.5 months, about once every 3 months, about once every 3.5 months, about once every 4 months, about once every 4.5 months, about once every 5 months, about once every 5.5 months, and about once every 6 months. Such an intervention treatment regimen may be recommended either alone or in conjunction with corticosteroids or other treatment outlined herein.

In one aspect, an intervention treatment regiment may include recommending sigmoidoscopy and/or the administration of budesonide to the patient either before, after or concurrent with said co-stimulatory pathway modulator. Such an intervention treatment regimen may be recommended either alone or in conjunction with corticosteroids or other treatment outlined herein.

In one aspect, an intervention treatment regiment may include recommending the administration of solumedrol and/or infliximab to the patient either before, after or concurrent with said co-stimulatory pathway modulator. Such an intervention treatment regimen may be recommended either alone or in conjunction with corticosteroids or other treatment outlined herein.

In one aspect, an intervention treatment regiment may include recommending the administration of an agonist CD137 antibody prior to the next scheduled administration of said co-stimulatory pathway modulator. Such an intervention treatment regimen may be recommended either alone or in conjunction with corticosteroids or other treatment outlined herein.

A therapeutically effective amount of co-stimulatory pathway modulator, preferably ipilimumab, can be orally administered if it is a small molecule modulator, for example, or preferably injected into the patient. The actual dosage employed can be varied depending upon the requirements of the patient and the severity of the condition being treated, including consideration of the expression level of CD177, CEACAM1, and/or other biomarker disclosed. Determination of the proper starting dosage for a particular situation is within the skill of the art, though the assignment of a treatment regimen will benefit from taking into consideration the expression level of CD177, CEACAM1, and/or other biomarker disclosed. Nonetheless, it will be understood that the specific dose level and frequency of dosing for any particular patient can be varied and will depend upon a variety of factors including the activity of the specific compound employed, the metabolic stability and length of action of that compound, the species, age, body weight, general health, sex and diet of the patient, the mode and time of administration, rate of excretion, drug combination, and severity of the particular condition. Preferred patients for treatment include animals, most preferably mammalian species such as humans, and domestic animals such as dogs, cats, and the like, patient to cancer.

The terms “combination” and “combinations” as used herein refer to a combination of a co-stimulatory pathway modulator, preferably an agonist, with another co-stimulatory pathway modulator, preferably an agonist (i.e., immunostimulant), or chemotherapeutic agents such as a tubulin stabilizing agent (e.g., pacitaxol, epothilone, taxane, etc.), Bevacizumab, IXEMPRA®, Dacarbazine, PARAPLATIN®, Docetaxel, one or more peptide vaccines, such as PROVENGE®, MDX-1379 Melanoma Peptide Vaccine, one or more gp100 peptide vaccine, fowlpox-PSA-Tricom vaccine, vaccinia-PSA-Tricom vaccine, MART-1 antigen, sargramostim, ticilimumab, Combination Androgen Ablative Therapy; the combination of ipilimumab and another co-stimulatory pathway modulator; combination of ipilimumab and a tubulin stabilizing agent (e.g., pacitaxol, epothilone, taxane, etc.); combination of ipilimumab and IXEMPRA® the combination of ipilimumab with Dacarbazine, the combination of ipilimumab with PARAPLATIN®, the combination of ipilimumab with Docetaxel, the combination of ipilimumab with one or more peptide vaccines, the combination of ipilimumab with MDX-1379 Melanoma Peptide Vaccine, the combination of ipilimumab with one or more gp100 peptide vaccine, the combination of ipilimumab with fowlpox-PSA-Tricom vaccine, the combination of ipilimumab with vaccinia-PSA-Tricom vaccine, the combination of ipilimumab with MART-1 antigen, the combination of ipilimumab with sargramostim, the combination of ipilimumab with ticilimumab, and/or the combination of ipilimumab with Combination Androgen Ablative Therapy. The combinations of the present invention may also be used in conjunction with other well known therapies that are selected for their particular usefulness against the condition that is being treated. Such combinations may provide therapeutic options to those patients who present with elevated expression levels of CD177, CEACAM1, and/or other biomarker disclosed subsequent to the administration of an anti-CTLA-4 antibody.

In another embodiment of the present invention, combination between a co-stimulatory pathway modulator and at least one other agent may comprise one or more of the following combinations: ipilimumab and TAXOL® and PARAPLATIN® (concurrent administration); ipilimumab and TAXOL® and PARAPLATIN® (sequential administration); ipilimumab and Dacarbazine; ipilimumab and Bevacizumab; ipilimumab and Budesonide; ipilimumab and an inhibitor of CD137; and ipilimumab and steroids (corticosteroids and the like).

Expression levels of CD177, CEACAM1, and/or other biomarker disclosed may be useful as a predictive indicator of patient's likelihood of developing a GI-irAE subsequent to the administration of other co-stimulatory pathway modulators alone, or in combination with other co-stimulatory pathway modulators disclosed herein, which include, but are not limited to, the following: agatolimod, belatacept, blinatumomab, CD40 ligand, anti-B7-1 antibody, anti-B7-2 antibody, anti-B7-H4 antibody, AG4263, eritoran, anti-CD137 monoclonal antibodies, anti-OX40 antibody, ISF-154, and SGN-70.

A variety of chemotherapeutics are known in the art, some of which are described herein. One type of chemotherapeutic is referred to as a metal coordination complex. It is believed this type of chemotherapeutic forms predominantly inter-strand DNA crosslinks in the nuclei of cells, thereby preventing cellular replication. As a result, tumor growth is initially repressed, and then reversed. Another type of chemotherapeutic is referred to as an alkylating agent. These compounds function by inserting foreign compositions or molecules into the DNA of dividing cancer cells. As a result of these foreign moieties, the normal functions of cancer cells are disrupted and proliferation is prevented. Another type of chemotherapeutic is an antineoplastic agent. This type of agent prevents, kills, or blocks the growth and spread of cancer cells. Still other types of anticancer agents include nonsteroidal aromastase inhibitors, bifunctional alkylating agents, etc.

Immunotherapy, in combination with chemotherapy, is a novel approach for the treatment of cancer which combines the effects of agents that directly attack tumor cells producing tumor cell necrosis or apoptosis, and agents that modulate host immune responses to the tumor. Chemotherapeutic agents could enhance the effect of immunotherapy by generating tumor antigens to be presented by antigen-presenting cells creating a “polyvalent” tumor cell vaccine, and by distorting the tumor architecture, thus facilitating the penetration of the immunotherapeutic agents as well as the expanded immune population.

The anti-CTLA-4 antibody may preferably be administered at about 0.3-10 mg/kg, or the maximum tolerated dose. In an embodiment of the invention, a dosage of CTLA-4 antibody is administered about every three weeks. Alternatively, the CTLA-4 antibody may be administered by an escalating dosage regimen including administering a first dosage of CTLA-4 antibody at about 3 mg/kg, a second dosage of CTLA-4 antibody at about 5 mg/kg, and a third dosage of CTLA-4 antibody at about 10 mg/kg.

In another specific embodiment, the escalating dosage regimen includes administering a first dosage of CTLA-4 antibody at about 5 mg/kg and a second dosage of CTLA-4 antibody at about 10 mg/kg.

Further, the present invention provides an escalating dosage regimen, which includes administering an increasing dosage of CTLA-4 antibody about every three weeks.

In an aspect of the present invention, a stepwise escalating dosage regimen is provided, which includes administering a first CTLA-4 antibody dosage of about 3 mg/kg, a second CTLA-4 antibody dosage of about 3 mg/kg, a third CTLA-4 antibody dosage of about 5 mg/kg, a fourth CTLA-4 antibody dosage of about 5 mg/kg, and a fifth CTLA-4 antibody dosage of about 10 mg/kg. In another aspect of the present invention, a stepwise escalating dosage regimen is provided, which includes administering a first dosage of 5 mg/kg, a second dosage of 5 mg/kg, and a third dosage of 10 mg/kg.

The actual dosage employed may be varied depending upon the requirements of the patient and the severity of the condition being treated or the occurrence of severe GI-irAEs, which may be determined by consideration of the expression level of CD177, CEACAM1, and/or other biomarker disclosed subsequent to the administration of an anti-CTLA-4 modulator in accordance with the present invention. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In practicing the many aspects of the invention herein, biological samples can be selected preferably from blood or blood cells (red blood cells or white blood cells). Cells from a sample can be used, or a lysate of a cell sample can be used. In certain embodiments, the biological sample comprises blood cells or mRNA derived from blood cells.

Pharmaceutical compositions for use in the present invention can include compositions comprising one or a combination of co-stimulatory pathway modulators in an effective amount to achieve the intended purpose. A therapeutically safe and effective dose refers to that amount of active ingredient which ameliorates the symptoms or condition in a safe mode, and should take into consideration the expression level of CD177, CEACAM1, and/or other biomarker disclosed subsequent to the administration of an anti-CTLA-4 modulator in accordance with the present invention. Therapeutic efficacy and toxicity in humans can be predicted by standard pharmaceutical procedures in cell cultures or experimental animals, for example the ED50 (the dose therapeutically effective in 50% of the population) and LD50 (the dose lethal to 50% of the population).

A “therapeutically effective amount” of a modulator of the co-stimulatory pathway can be a function of whether a patient exhibits elevated or decreased expression level of CD177, CEACAM1, and/or other biomarker disclosed herein subsequent to the administration of an anti-CTLA-4 modulator. A therapeutically relevant dose of a co-stimulatory pathway modulator for patients having an elevated expression level of CD177, CEACAM1, and/or other biomarker disclosed herein, for example, could range anywhere from 1 to 14 fold or more lower than the typical dose. Accordingly, therapeutically relevant doses of a co-stimulatory pathway modulator, such as ipilimumab, for any disorder disclosed herein, preferably melanoma, can be, for example, about 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 fold lower than the prescribed or standard dose. Alternatively, therapeutically relevant doses of a co-stimulatory pathway modulator, such as ipilimumab, can be, for example, about 1.0×, about 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01× of the prescribed dose for individuals exhibiting an elevated expression level of CD177, CEACAM1, and/or other biomarker disclosed herein. Alternatively, therapeutically relevant doses of a co-stimulatory pathway modulator, such as ipilimumab, can be, for example, about 1.0×, about 1.25×, 1.5×, 2×, 2.5×, 3×, 3.5, 4×, or even 5× more of the prescribed dose for individuals exhibiting the same or decreased expression level of CD177, CEACAM1, and/or other biomarker disclosed herein subsequent to the administration of an anti-CTLA-4 modulator.

Disorders for which an anti-CTLA-4 modulator may be useful for treating patients beyond merely melanoma, prostate cancer, and lung cancer, for example, also include leukemias, including, for example, chronic myeloid leukemia (CML), acute lymphoblastic leukemia, and Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkins lymphoma, hairy cell lymphoma and Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ ALL, AML, imatinib-resistant CML, imatinib-intolerant CML, accelerated CML, lymphoid blast phase CML.

The terms “cancer”, “cancerous”, or “malignant” refer to or describe the physiological condition in mammals, or other organisms, that is typically characterized by unregulated cell growth. Examples of cancer include, for example, solid tumors, melanoma, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

A “solid tumor” includes, for example, sarcoma, melanoma, colon carcinoma, breast carcinoma, prostate carcinoma, or other solid tumor cancer.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ ALL).

### Antibodies

Expression level of CD177, CEACAM1, and/or other biomarkers disclosed herein may be useful as a predictive indicator of patient's likelihood of developing a GI-irAE subsequent to the administration of antibodies that can specifically bind to co-stimulatory pathway polypeptides, such as CTLA-4, CD28, CD80, and CD86 among other modulators. The term “antibody” is used in the broadest sense and specifically covers monoclonal antibodies, polyclonal antibodies, antibody compositions with polyepitopic specificity, bispecific antibodies, diabodies, chimeric, single-chain, and humanized antibodies, as well as antibody fragments (e.g., Fab, F(ab′)₂, and Fv), so long as they exhibit the desired biological activity. Antibodies can be labeled for use in biological assays (e.g., radioisotope labels, fluorescent labels) to aid in detection of the antibody.

Antibodies that bind to co-stimulatory pathway polypeptides can be prepared using, for example, intact polypeptides or fragments containing small peptides of interest, which can be prepared recombinantly for use as the immunizing antigen. The polypeptide or oligopeptide used to immunize an animal can be derived from the translation of RNA or synthesized chemically, and can be conjugated to a carrier protein, if desired. Commonly used carriers that are chemically coupled to peptides include, for example, bovine serum albumin (BSA), keyhole limpet hemocyanin (KLH), and thyroglobulin. The coupled peptide is then used to immunize the animal (e.g., a mouse, a rat, or a rabbit).

The term “antigenic determinant” refers to that portion of a molecule that makes contact with a particular antibody (i.e., an epitope). When a protein or fragment of a protein is used to immunize a host animal, numerous regions of the protein can induce the production of antibodies that bind specifically to a given region or three-dimensional structure on the protein; each of these regions or structures is referred to as an antigenic determinant. An antigenic determinant can compete with the intact antigen (i.e., the immunogen used to elicit the immune response) for binding to an antibody.

The phrase “specifically binds to” refers to a binding reaction that is determinative of the presence of a target in the presence of a heterogeneous population of other biologics. Thus, under designated assay conditions, the specified binding region binds preferentially to a particular target and does not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice background signal or noise and more typically more than 10 times background. For purposes of the present invention, compounds, for example small molecules, can be considered for their ability to specifically bind to co-stimulatory pathway polypeptides described herein.

### Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise a means for measuring the expression level of CD177, CEACAM1, and/or other biomarker disclosed herein, on a patient sample and/or instructions for interpreting the measurement value obtained. Another example of a container means can comprise one or more vials containing a pharmaceutically acceptable amount of a co-stimulatory pathway modulator.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

Kits useful in practicing therapeutic methods disclosed herein can also contain a compound that is capable of inhibiting the co-stimulatory pathway. Specifically contemplated by the invention is a kit comprising an anti-CTLA-4 antibody, either alone or in combination with another immunotherapy agent, such as a tubulin stabilizing agent (e.g., pacitaxol, epothilone, taxane, etc.); a peptide vaccine such as PROVENGE®; and/or a second co-stimulatory pathway modulator, such as, tremelimumab. In addition, contemplated by the invention is a kit comprising an a different dose and/or dosing frequency regimen of a co-stimulatory pathway modulator, and any other combination or dosing regimen comprising a tubulin stabilizing agent (e.g., pacitaxol, epothilone, taxane, etc.); and/or a second co-stimulatory pathway modulator, such as, tremelimumab.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### Biomarkers and Biomarker Sets

The invention includes individual biomarkers and biomarker sets having both diagnostic and prognostic value in predicting the likelihood a patient may be at an increased risk of developing a gastrointestinal immune-related adverse event subsequent to the administration of an anti-CTLA-4 modulator. The biomarker sets may comprise one or a plurality of biomarkers that highly correlate with the incidence of a GI-irAE.

The biomarkers and biomarker sets of the invention enable one to predict or reasonably foretell the likelihood some patients may be at an increased risk of developing a gastrointestinal immune-related adverse event subsequent to the administration of an anti-CTLA-4 modulator based upon whether one or more of the biomarkers of the present invention are overexpressed relative to a baseline, predetermined or normal level. The biomarkers and biomarker sets can be used in in vitro assays by measuring the expression levels in a patient sample to predict in vivo outcome. In accordance with the invention, the various biomarkers and biomarker sets described herein, or the combination of these biomarker sets with other biomarkers or markers, can be used, for example, to also monitor the likelihood of a patient developing a gastrointestinal immune-related adverse event over time by analyzing whether said expression increases over time, in addition to whether or not said expression surpasses a GI-irAE threshold, subsequent to the administration of an anti-CTLA-4 modulator.

Measuring the level of expression of a biomarker and biomarker set provides a useful tool for screening one or more blood samples before and/or subsequent to treatment of a patient with an anti-CTLA-4 modulator.

A difference in the level of the biomarker that is sufficient to indicate whether the mammal will or will develop GI-irAE after receiving cancer treatment can be readily determined by one of skill in the art using known techniques. The increase or decrease in the level of the biomarker subsequent to the administration of a CTLA-4 antagonist can be correlated to determine whether the difference is sufficient to increase the likelihood a patient will develop a gastrointestinal immune-related adverse event. In one aspect, the difference in the level of the biomarker is a difference in the mRNA level (measured, for example, by RT-PCR or a microarray), such as at least about a two-fold difference, at least about a three-fold difference, or at least about a four-fold difference in the level of expression, or more. In another aspect, the difference in the level of the biomarker is determined at the protein level by mass spectral methods or by FISH or by IHC. In another aspect, the difference in the level of the biomarker refers to a p-value of <0.05 in Anova analysis. In yet another aspect, the difference is determined in an ELISA assay.

The biomarker or biomarker set(s) outlined herein can also be used as described herein for monitoring the progress of disease treatment or therapy in those patients undergoing treatment for a disease involving an anti-CTLA-4 treatment.

The biomarkers also serve as targets for the development of therapies for immune related adverse event subsequent to cancer treatment. Such targets may be particularly applicable to treatment of GI-irAE subsequent to treatment of cancers, such as, for example, melanoma, prostate cancer, lung cancer, non-small cell lung cancer, and small cell lung cancer, among other cancers outlined herein.

The level of biomarker protein and/or mRNA can be determined using methods well known to those skilled in the art. For example, quantification of protein can be carried out using methods such as ELISA, 2-dimensional SDS PAGE, Western blot, immunoprecipitation, immunohistochemistry, fluorescence activated cell sorting (FACS), or flow cytometry. Quantification of mRNA can be carried out using methods such as PCR, array hybridization, Northern blot, in-situ hybridization, dot-blot, TAQMAN®, or RNAse protection assay.

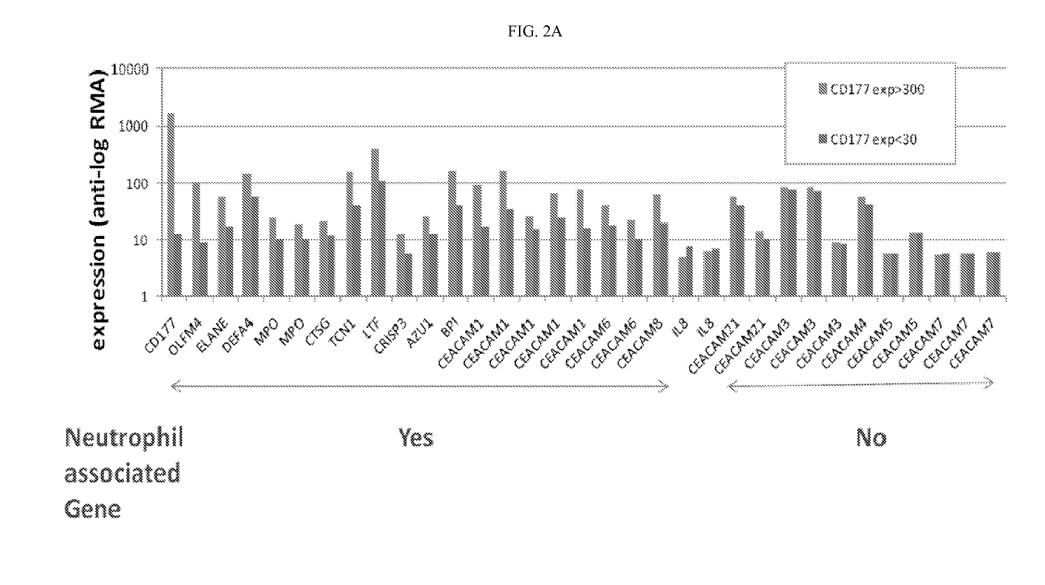

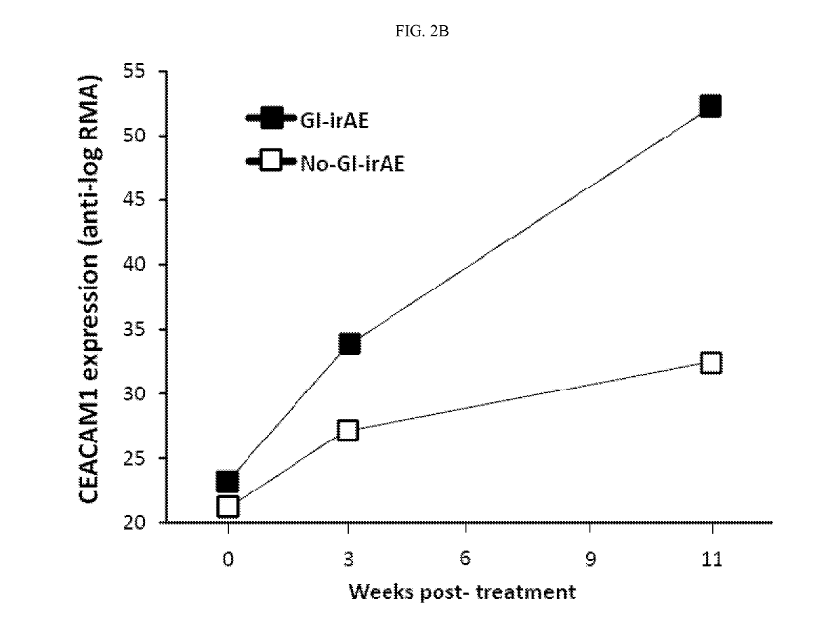

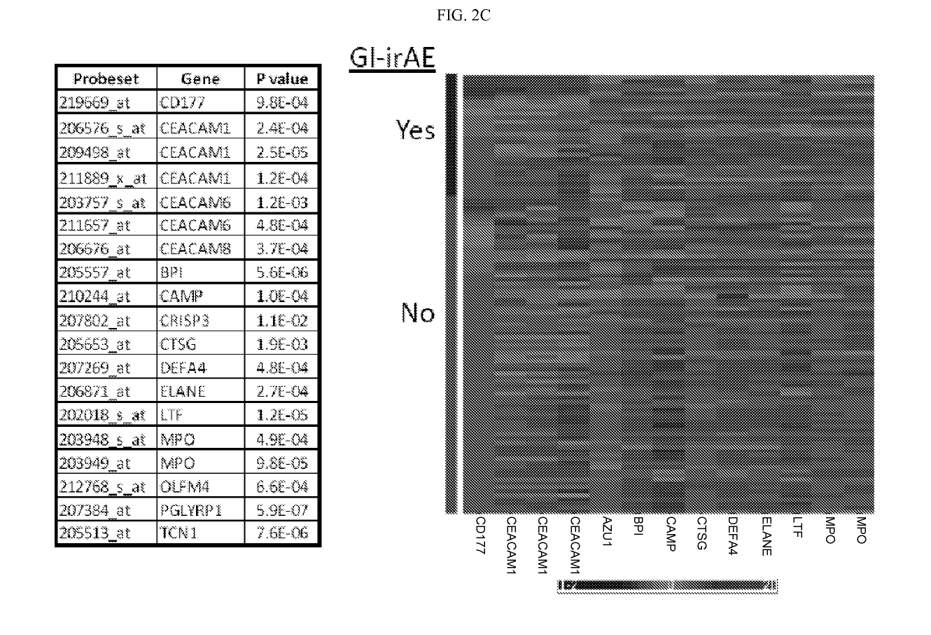

The present invention encompasses the use of any one or more of the following as a biomarker for use in predicting the likelihood a patient may develop a GI-irAE subsequent to the administration of an anti-CTLA-4 modulator: CD177, and CEACAM1, in addition to the biomarkers disclosed in FIGS. 2A and 2C, Table 1A-B, and Table 3, and elsewhere herein.

Identification of biomarkers that provide rapid and accessible readouts of efficacy, adverse events, drug exposure, or clinical response is increasingly important in the clinical development of drug candidates. Embodiments of the invention include measuring changes in the levels of mRNA and/or protein in a sample to determine whether said sample contains increased expression of CD177 and/or CEACAM1. In one aspect, said samples serve as surrogate tissue for biomarker analysis. These biomarkers can be employed for predicting and monitoring a patient's likelihood of developing a gastrointestinal immune-related adverse event subsequent to treatment with an anti-CTLA antagonist. In one aspect, the biomarkers of the invention are one or more of the following: CD177 and/or CEACAM1, including both polynucleotide and polypeptide sequences. In another aspect, the biomarkers of the invention are nucleotide sequences that, due to the degeneracy of the genetic code, encodes for a polypeptide sequence provided in the sequence listing.

Methods of measuring the level of any given marker described herein may be performed using methods well known in the art, which include, but are not limited to PCR; RT-PCR; FISH; IHC; immunodetection methods; immunoprecipitation; Western Blots; ELISA; radioimmunoassays; FACS; HPLC; surface plasmon resonance, and optical spectroscopy; and mass spectrometry, among others.

The biomarkers of the invention may be quantified using any immunospecific binding method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., Current Protocols in Molecular Biology, Vol. 1, John Wiley & Sons, Inc., New York (1994), which is incorporated by reference herein in its entirety). Exemplary immunoassays are described briefly below (but are not intended by way of limitation).

The biomarkers of the invention may be quantified using any qPCR analysis known in the art. Such analysis can be done by various methods such as but not limited to two-step qPCR assay which can be performed to determine changes in the expression levels of target genes. Total RNA is reverse transcribed into cDNA using the SUPERSCRIPT® Vilo cDNA synthesis kit. The cDNA samples are mixed with ABI 2× TAQMAN® Universal PCR Master Mix and loaded into the TAQMAN® Low Density Array Card. A 384-well micro fluidiccard, pre-loaded with target genes and 2 housekeeping probe sets (ABI Assay-on-Demand TAQMAN® Gene Expression Assays, ABI, P/N 4342249) is run on ABI 7900HT Systems for relative quantitation according to manufacturer's instructions. The expression data can be normalized using ACT method with 18S as housekeeping gene.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% TRASYLOL®) supplemented with protein phosphatase and/or protease inhibitors (e.g., EDTA, PMSF, aprotinin, sodium vanadate), adding the antibody of interest (i.e., one directed to a biomarker of the present invention) to the cell lysate, incubating for a period of time (e.g., 1-4 hours) at 4° C., adding protein A and/or protein G SEPHAROSE® beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with SEPHAROSE® beads). For further discussion regarding immunoprecipitation protocols see, e.g., Ausubel et al., eds., Current Protocols in Molecular Biology, Vol. 1, p. 10.16.1, John Wiley & Sons, Inc., New York (1994).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds., Current Protocols in Molecular Biology, Vol. 1, p. 10.8.1, John Wiley & Sons, Inc., New York (1994).

ELISAs comprise preparing antigen, coating the well of a 96-well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds., Current Protocols in Molecular Biology, Vol. 1, p. 11.2.1, John Wiley & Sons, Inc., New York (1994).

Alternatively, identifying the relative quantitation of the biomarker polypeptide(s) may be performed using tandem mass spectrometry; or single or multi dimensional high performance liquid chromatography coupled to tandem mass spectrometry. The method takes into account the fact that an increased number of fragments of an identified protein isolated using single or multi dimensional high performance liquid chromatography coupled to tandem mass spectrometry directly correlates with the level of the protein present in the sample. Such methods are well known to those skilled in the art and described in numerous publications, for example, Link, A. J., ed., 2-D Proteome Analysis Protocols, Humana Press (1999), ISBN: 0896035247; Chapman, J. R., ed., Mass Spectrometry of Proteins and Peptides, Humana Press (2000), ISBN: 089603609X.

As used herein the terms “modulate” or “modulates” or “modulators” refer to an increase or decrease in the amount, quality or effect of a particular activity, or the level of DNA, RNA, or protein detected in a sample.

In order to facilitate a further understanding of the invention, the following examples are presented primarily for the purpose of illustrating more specific details thereof. The scope of the invention should not be deemed limited by the examples, but to encompass the entire subject matter defined by the claims.

### BRIEF DESCRIPTION OF THE SEQUENCE LISTING

Incorporated herein by reference in its entirety is a Sequence Listing, comprising SEQ ID NO:1 through SEQ ID NO:4, which include nucleic acid and amino acid sequences of the biomarkers presented herein. The Sequence Listing is contained on a compact disc, i.e., CD-ROM, three identical copies of which are filed herewith. The Sequence Listing, in IBM/PC MS-DOS (ASCII) text format, was first created on Mar. 15, 2012, and is 2.5 KB in size.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope.

### REFERENCES

1. Korman, A. J. et al., “Checkpoint blockade in cancer immunotherapy”, Adv. Immunol., 90:297-339 (2006).
2. Hodi, F. S. et al., “Improved survival with ipilimumab in patients with metastatic melanoma”, New Engl. J. Med., 363:711-723 (2010).
3. Weber, J., “Overcoming immunologic tolerance to melanoma: targeting CTLA-4 with ipilimumab (MDX-010)”, Oncologist, 13(Suppl. 4):16-25 (2008).
4. Beck, K. E. et al., “Enterocolitis in patients with cancer after antibody blockade of cytotoxic T-lymphocyte-associated antigen 4”, J. Clin. Oncol., 24:2283-2289 (2006).
5. Berman, D. et al., “Blockade of cytotoxic T-lymphocyte antigen-4 by ipilimumab results in dysregulation of gastrointestinal immunity in patients with advanced melanoma”, Cancer Immun., 10:11 (2010).
6. Weber, J. et al., “A randomized, double-blind, placebo-controlled, phase II study comparing the tolerability and efficacy of ipilimumab administered with or without prophylactic budesonide in patients with unresectable stage III or IV melanoma”, Clin. Cancer Res., 15:5591-5598 (2009).
7. Minor, D. R. et al., “Infliximab in the treatment of anti-CTLA-4 antibody (ipilimumab) induced immune-related colitis”, Cancer Biother. Radiopharm., 24:321-325 (2009).
8. Hamid, O. et al., “Association of baseline and on-study tumor biopsy markers with clinical activity in patients (pts) with advanced melanoma treated with ipilimumab”, AJ Clin. Oncol., 27(Suppl.), Abstract 9008 (2009).
9. Hamid, O. et al., “A prospective phase II trial exploring the association between tumor microenvironment biomarkers and clinical activity of ipilimumab in advanced melanoma”, J. Transl. Med., 9:204 (2011).
10. Weber, J. et al., “Assessment of pharmacokinetic interaction between ipilimumab and chemotherapy in a randomized study”, 35th ESMO Congress Abstract 1329P (2010).
11. Ji, R. R. et al., “An immune-active tumor microenvironment favors clinical response to ipilimumab”, Cancer Immunol. Immunother., 61(7):1019-1031 (2011).
12. Wang, X. et al., “Structure of the quaternary complex of interleukin-2 with its alpha, beta, and gammac receptors”, Science, 310(5751):1159-1163 (2005).
13. Zhao, X. et al., “Targeting cd37-positive lymphoid malignancies with a novel engineered small modular immunopharmaceutical”, Blood, 110(7):2569-2577 (2007).
14. Kim, S. H. et al., “Interleukin-32: A cytokine and inducer of tnfalpha”, Immunity, 22(1):131-142 (2005).
15. Zhang, H. et al., “Human neutrophils coordinate chemotaxis by differential activation of rac1 and rac2”, J. Immunol., 183(4):2718-2728 (2009).
16. McMahon, L. W. et al., “Human alpha spectrin ii and the fanconi anemia proteins fanca and fancc interact to form a nuclear complex”, J. Biol. Chem., 274(46):32904-32908 (1999).
17. Segura-Totten, M. et al., “Baf: Roles in chromatin, nuclear structure and retrovirus integration”, Trends Cell Biol., 14(5):261-266 (2004).
18. Peelman, L. J. et al., “The bat1 gene in the mhc encodes an evolutionarily conserved putative nuclear ma helicase of the dead family”, Genomics, 26(2):210-218 (1995).
19. Gearhart, M. D. et al., “Polycomb group and scf ubiquitin ligases are found in a novel bcor complex that is recruited to bcl6 targets”, Mol. Cell. Biol., 26(18):6880-6889 (2006).
20. Lee, Y. S. et al., “Molecular signature of clinical severity in recovering patients with severe acute respiratory syndrome coronavirus (sars-cov)”, BMC Genomics, doi: 10.1186/1471-2164-6-132 (Sep. 21, 2005).

21. Fujibuchi, T. et al., “Aip1/wdr1 supports mitotic cell rounding”, Biochem. Biophys. Res. Comm., 327(1):268-275 (2005).
22. Tebar, F. et al., “Clathrin assembly lymphoid myeloid leukemia (calm) protein: Localization in endocytic-coated pits, interactions with clathrin, and the impact of overexpression on clathrin-mediated traffic”, Mol. Biol. Cell, 10(8):2687-2702 (1999).
23. Bernstein, A. M. et al., “Identification of a cellubrevin/vesicle associated membrane protein 3 homologue in human platelets”, Blood, 93(2):571-579 (1999).
24. Lalezari, P. et al. “NB 1, a new neutrophil-specific antigen involved in the pathogenesis of neonatal neutropenia”, J. Clin. Invest., 50:1108-1115 (1971).
25. Stroncek, D. F. et al., “Analysis of the expression of NB1 antigen using two monoclonal antibodies”, Transfusion, 36:168-174 (1996).
26. Stroncek, D. F. et al., “Biochemical characterization of the neutrophil-specific antigen NB1”, Blood, 75:744-755 (1990).
27. Rosenbauer, F. et al., “pDP4, a novel glycoprotein secreted by mature granulocytes, is regulated by transcription factor PU.1”, Blood, 103:4294-4301 (2004).
28. Miyauchi, J. et al., “Immunocytochemical localization of lactoferrin in human neutrophils. An ultrastructural and morphometrical study”, Cell Tissue Res., 247:249-258 (1987).
29. Falloon, J. et al., “Neutrophil granules in health and disease”, J. Allergy Clin. Immunol., 77:653-662 (1986).
30. Gabay, J. E. et al., “Antibiotic proteins of human polymorphonuclear leukocytes”, Proc. Natl. Acad. Sci. USA, 86:5610-5614 (1989).
31. Korkmaz, B. et al., “Neutrophil elastase, proteinase 3, and cathepsin G as therapeutic targets in human diseases”, Pharmacol. Rev., 62:726-759 (2010).
32. Zhao, L. et al., “Subcellular localization and mobilization of carcinoembryonic antigen-related cell adhesion molecule 8 in human neutrophils”, Br. J. Haematol., 125:666-673 (2004).
33. Skubitz, K. M. et al., “Interdependency of ceacam-1, -3, -6, and -8 induced human neutrophil adhesion to endothelial cells”, J. Transl. Med., 6:78 (2008).
34. Chaussabel, D. et al., “A modular analysis framework for blood genomics studies: Application to systemic lupus erythematosus”, Immunity, 29(1):150-164 (2008).
35. Robert, C. et al., “Ipilimumab plus dacarbazine for previously untreated metastatic melanoma”, New Engl. J. Med., 364(26):2517-2526 (2011).
36. Shioya, M. et al., “Epithelial overexpression of interleukin-32alpha in inflammatory bowel disease”, Clin. Exp. Immunol., 149(3):480-486 (2007).
37. Cagnard, N. et al., “Interleukin-32, cc12, pf4f1 and gfd10 are the only cytokine/chemokine genes differentially expressed by in vitro cultured rheumatoid and osteoarthritis fibroblast-like synoviocytes”, European Cytokine Network, 16(4):289-292 (2005).
38. Monsurro, V. et al., “Quiescent phenotype of tumor-specific cd8+t cells following immunization”, Blood, 104(7):1970-1978 (2004).
39. Jaeschke, H. et al., “Mechanisms of inflammatory liver injury: Adhesion molecules and cytotoxicity of neutrophils”, Toxicol. Appl. Pharmacol., 139(2):213-226 (1996).
40. Segura, R. M. et al., “Interleukin-8 and markers of neutrophil degranulation in pleural effusions”, Am. J. Respir. Crit. Care Med., 157(5 Pt. 1):1565-1572 (1998).
41. Jacobs, L. et al., “Subclinical responses in healthy cyclists briefly exposed to traffic-related air pollution: An intervention study”, Environ. Health, doi: 10.1186/1476-069X-9-64 (published online Oct. 25, 2010).

42. De Larco, J. E. et al., “The potential role of neutrophils in promoting the metastatic phenotype of tumors releasing interleukin-8”, Clin. Cancer Res., 10:4895-4900 (2004).
43. Waugh, D. J. et al., “The interleukin-8 pathway in cancer”, Clin. Cancer Res., 14:6735-6741 (2008).
44. Follin, P. et al., “Human neutrophil migration into skin chambers is associated with production of NAP-1/IL8 and C5a”, Eur. J. Haematol., 47:71-76 (1991).
45. Gray-Owen, S. D. et al., “Ceacam1: Contact-dependent control of immunity”, Nature Rev., 6(6):433-446 (2006).
46. Gohring, K. et al., “Neutrophil CD177 (NB1 gp, HNA-2a) expression is increased in severe bacterial infections and polycythaemia vera”, Br. J. Haematol., 126:252-254 (2004).
47. Skubitz, K. M. et al., “Two new synthetic peptides from the N-domain of CEACAM1 (CD66a) stimulate neutrophil adhesion to endothelial cells”, Biopolymers, 96:25-31 (2011).
48. Costello, C. M. et al., “Dissection of the inflammatory bowel disease transcriptome using genome-wide cDNA microarrays”, PLoS Med., 2:e199 (2005).
49. Sachs, U. J. et al., “The neutrophil-specific antigen CD177 is a counter-receptor for platelet endothelial cell adhesion molecule-1 (CD31)”, J. Biol. Chem., 282:23603-23612 (2007).
50. Witko-Sarsat, V. et al., “Neutrophils: molecules, functions and pathophysiological aspects”, Lab. Invest., 80:617-653 (2000).
51. Kabakchiev, B. et al., “Gene expression changes associated with resistance to intravenous corticosteroid therapy in children with severe ulcerative colitis”, PLoS One, 5(9):e13085 (2010).
52. Merlo, A. et al., “Inhibitory receptors cd85j, lair-1, and cd152 down-regulate immunoglobulin and cytokine production by human b lymphocytes”, Clin. Diagn. Lab. Immunol., 12(6):705-712 (2005).

53. Zitvogel, L. et al., “Integration of host-related signatures with cancer cell-derived predictors for the optimal management of anticancer chemotherapy”, Cancer Res., 70:9538-9543 (2010).

### EXAMPLES

### Example 1

### Methods Used to Identify Biomarkers for Predicting Gastrointestinal Immuned-Related Adverse Events in Patients Administered Costimulatory Pathway Inhibitors

CTLA-4 is a negative regulator of the activation of T cell lymphocytes. The efficacy of ipilimumab has been demonstrated in a number of phase II and two phase III clinical trials in metastatic melanoma patients, where a prolongation of overall survival has been reported (Hodi, F. S. et al., New Engl. J. Med., 363:711-723 (2010); Weber, J. et al., Oncologist, 13(Suppl. 4):16-25 (2008)).

As a consequence of the CTLA-4 blockade, treatment with ipilimumab is associated with a number of immune related adverse events (irAEs). Gastrointestinal (GI) dysregulations such as diarrhea and colitis are of the most common ipilimumab associated irAEs (Beck, K. E. et al., J. Clin. Oncol., 24:2283-2289 (2006)). In most cases the onset of GI-irAEs occur after the second or third dose of ipilimumab (Berman, D. et al., Cancer Immun., 10:11 (2010)). Examination of colonic biopsies in a safety-focused clinical trial (CA184007) revealed abundant focal neutrophilic cryptitis and neutrophilic infiltration in the lamina propria of affected tissues from patients experiencing GI-irAE. Although administration of high doses of steroids lead to successful and safe management of the majority of these irAEs (Berman, D. et al., Cancer Immun., 10:11 (2010); Weber, J. et al., Clin. Cancer Res., 15:5591-5598 (2009); Minor, D. R. et al., Cancer Biother. Radiopharm., 24:321-325 (2009)), identification of biomarkers that may predict (before or soon after the start of the treatment) these irAEs are crucial and can highly improve patient care. In this context, peripheral blood biomarkers are preferred as collection of these samples is less invasive than colonic biopsies.

To understand the underlying causes of ipilimumab associated GI-irAEs and identify potential predictive biomarkers that could help manage these irAEs, gene expression analysis was performed on whole blood (WB) samples collected from metastatic melanoma patients before and after ipilimumab treatment. Data from two phase II clinical trials (CA184004 and CA184007) (Weber, J. et al., Clin. Cancer Res., 15:5591-5598; Hamid, O. et al., AJ Clin. Oncol., 27(Suppl.), Abstract 9008 (2009)) were used in the analyses. The methods utilized are outlined as follows.

### Methods

Study Design. The multicenter, phase II clinical trial CA184-004 enrolled 82 previously-treated and untreated patients with unresectable stage III or IV melanoma, randomized 1:1 into 2 arms to receive up to 4 intravenous infusions of either 3 or 10 mg/kg ipilimumab every 3 weeks (Q3W) in the induction phase. In CA184-007 trial, treatment-naïve or previously treated patients with unresectable stage III/IV melanoma (N=115) received open-label ipilimumab (10 mg/kg every 3 wks for four doses) and were randomized to receive concomitant blinded prophylactic oral budesonide (9 mg/d with gradual taper through week 16) or placebo (Beck, K. E. et al., J. Clin. Oncol., 24:2283-2289 (2006)). The exclusion criteria included the use of any immunosuppressing treatments including corticosteroids (patients on stable doses of hormone replacement therapy were exempt), cyclosporine, mycophenolate mofetil (CELLCEPT®), chemotherapy, radiation, etc, within 4 weeks prior to Day 1 of treatment. Complete study design, patient characteristics and endpoint reports of these trials have been described elsewhere (Weber, J. et al., Clin. Cancer Res., 15:5591-5598 (2009); Hamid, O. et al., AJ Clin. Oncol., 27(Suppl.), Abstract 9008 (2009)). Both studies were conducted in accordance with the ethical principles originating from the current Declaration of Helsinki and consistent with International Conference on Harmonization Good Clinical Practice and the ethical principles underlying European Union Directive 2001/20/EC and the United States Code of Federal Regulations, Title 21, Part 50 (21 C.F.R. 50). The protocols and patient informed consent forms received appropriate approval by all Institutional Review Boards or Independent Ethics Committees prior to study initiation. All participating patients (or their legally acceptable representatives) gave written informed consent for these biomarker focused studies.

Adverse Event Evaluation. Safety was evaluated using the National Cancer Institute Common Terminology Criteria for Adverse Events, based on adverse events (AEs), physical examinations, and clinical laboratory assessments. Drug-related gastrointestinal AEs that were consistent with immune-mediated events and with the intrinsic biological activity of ipilimumab were examined and reported. An independent data monitoring committee reviewed safety data. Adverse events (AEs) were recorded based on MedDRA v10.0 system organ class (SOC) and Preferred Terms (PT) and listed by total AEs, serious AEs, AEs leading to ipilimumab discontinuation, and irAEs. Deaths within 70 days (5 ipilimumab half-lives) after last dose and cause of death were recorded. Clinical activity (CA) was defined as confirmed complete response, confirmed partial response, or stable disease ending not earlier than 24 weeks from date of first ipilimumab dose. Complete description of irAE evaluations for these trials has been reported elsewhere (Weber, J. et al., Clin. Cancer Res., 15:5591-5598 (2009); Hamid, O. et al., AJ Clin. Oncol., 27(Suppl.), Abstract 9008 (2009)).

Absolute Peripheral Blood Neutrophil Count (APBNC). Neutrophils were quantified in all patients randomized in the clinical studies CA184004 and CA184007 as a component of the standard hematology panel. Absolute neutrophil counts were available at various time points from most patients.

Affymetrix Gene Expression Analysis. Total RNA was extracted using the Prism 6100 (Applied Biosystems, Foster City, Calif.), purified by RNAClean Kit (Agencourt Bioscience Corporation; Beverly, Mass.), and evaluated on a 2100 Bioanalyzer (Agilent Technologies, Santa Clara, Calif.). Complementary DNA preparation and hybridization on HT-HG-U133A 96-array plates followed manufacturer's protocols (Affymetrix, Santa Clara, Calif.). The CEL files were analyzed with the robust multi-array analysis (RMA) algorithm, obtained from the bioconductor(.org) website. Appropriate Affymetrix control probe sets were examined to ensure quality control for the cDNA synthesis and the hybridization step. Principal component analysis (PCA) was subsequently performed to detect outlier samples (single samples that account for a high degree of variation in the data). No sample was removed as outlier. Anti-log RMA values were used in statistical analyses. For the combined data from studies CA184004 and CA184007, 12518 of the 22215 non-control probe sets had maximum expression level (RMA normalized) of less than 32. These probe sets, with low expression levels across all samples, were excluded from further analysis.

Gene Expression Analyses. GI-irAE status was available for all 197 treated patients. Gene expression data were available for 188 of these patients. Pooled gene expression data of whole blood samples were available for 162 out of 197 patients randomized in these two trials for at least two of three time points. Evaluable data on GI-irAE status for 162 patients were included in the statistical analyses. Of these patients, 113 were classified in the No-GI-irAE group. The status of GI-irAE in this group was grade 0 or 1 (G0-1). A total of 49 patients, who presented a status of grade 2 or greater (G2+) were classified in the GI-irAE group. Gene expression data were available for baseline (N=108 No-GI-irAE vs. N=49 GI-irAE), Week 3 (N=108 No-GI-irAE vs. N=47 GI-irAE) and Week 11 (N=78 No-GI-irAE N vs. N=30 GI-irAE).

A repeated measures analysis of variance (ANOVA) model was fit in PARTEK® Genomics Suite 6.6 (Partek), with anti-log normalized expression level as dependent variable. Explanatory variables included patient, time point within patient as a 3-level factor, and binary GI-irAE status, with no time-by-status interaction. Because GI-irAE was observed in all treatment arms, and because of the relatively small sample size, data from patients in the two trials were combined to increase the statistical power to detect associations. Statistical inference based on this model focused on two hypothesis tests: a test of the null hypothesis that mean gene expression (averaged over time) was the same in the two GI-irAE status groups, and a test of the null hypothesis that mean gene expression (averaged over GI-irAE status) was the same for the three time points. An uncorrected P value of 0.05 was used as a cutoff to select probe sets with mean expression differences between comparison groups. The qvalue package (v1.20.0) in the R statistical computing environment (v2.15.0, r-project) was used to estimate false discovery rates (FDRs). Expression of selected genes was confirmed by quantitative polymerase chain reaction (qPCR) as described previously using pre-designed probes (Ji et al., Cancer Immunol. Immunother., 61(7):1019-1031 (2011)).

Pathway Analysis. Gene or probe set lists were analyzed using the Ingenuity Pathway Analysis (IPA) software (Ingenuity), which performs a gene set enrichment analysis on the input lists of regulated probesets. The analysis was performed at the gene level and generated a P value for each functional category or canonical pathway based on Fisher's exact test. The P value reflects the significance of the enrichment of input genes in the functional category or pathway of interest. For every canonical pathway, IPA also provides the ratio of the number of genes from the input list that are annotated to the pathway to the total number of genes annotated to the pathway.

### Results

Gene Expression Profile of Pre-Treatment Samples. Expression profiles of whole blood samples from two clinical studies (CA184004 and CA184007) were pooled to increase the power of profiling analysis. Expression of each of 9697 non-control probe sets was analyzed individually. Genes associated with GI-irAE status (grade 2+ vs. not) were selected by assessing the difference in mean pre-treatment expression between the GI and No-GI-irAE groups. Two selection criteria were applied: a P value≦0.05 for the hypothesis test comparing the GI and No-GI-irAE groups, and a minimum mean pre-treatment expression ratio of 1.5. For these tests, the P value threshold of 0.05 corresponded to an estimated FDR of 0.50. A set of 27 probe sets representing 24 unique genes met these criteria (Table 1A). This list included a number of immune related genes including CD3E, IL2RG, CD37, CD4, IL32, and RAC2; cell cycle and cell proliferation associated genes such as SPTAN1, BANF1, BAT1, PCGF1, FP36L2, and WDR1; and intracellular vesicle trafficking such as PICALM, SNAP23, VAMP3. Some of these molecules such, as IL32, SNAP23 and RAC2, have been reported either to be present in neutrophils or to regulate their function. The majority of these probe sets (21 out of 27) also showed higher expression in blood samples from patients experiencing GI-irAEs taken at any of the two on-treatment time points (week 3 or 11), confirming their significance as biomarkers associated with GI-irAEs (Table 1B).

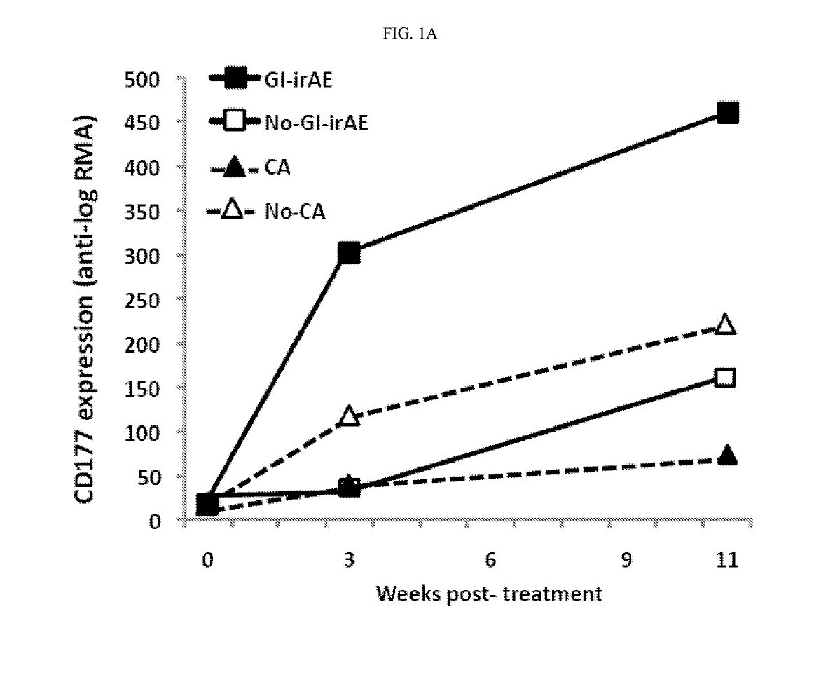

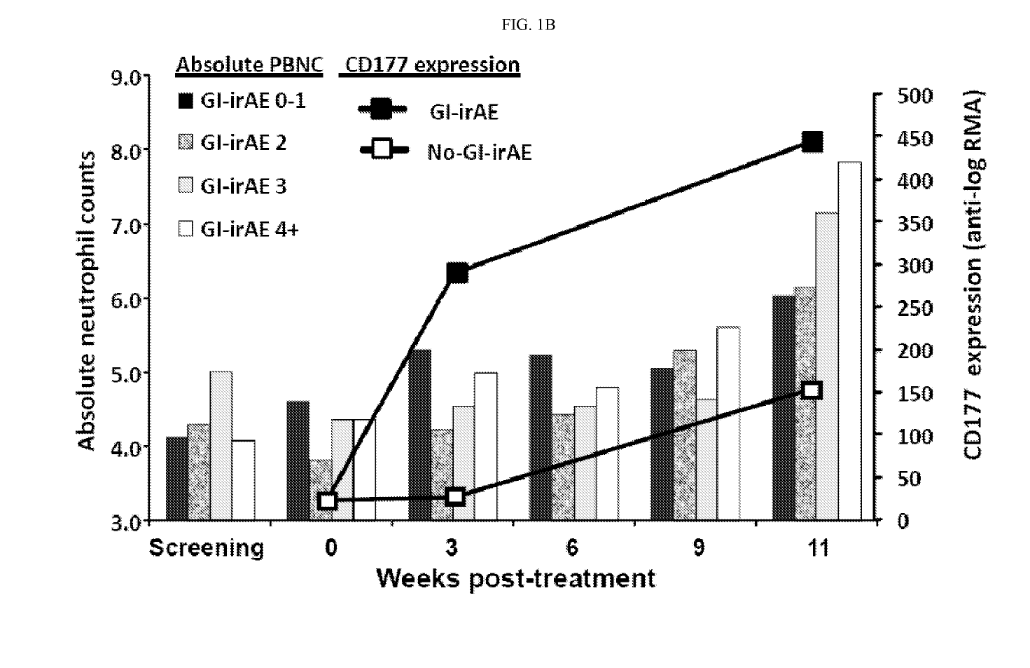

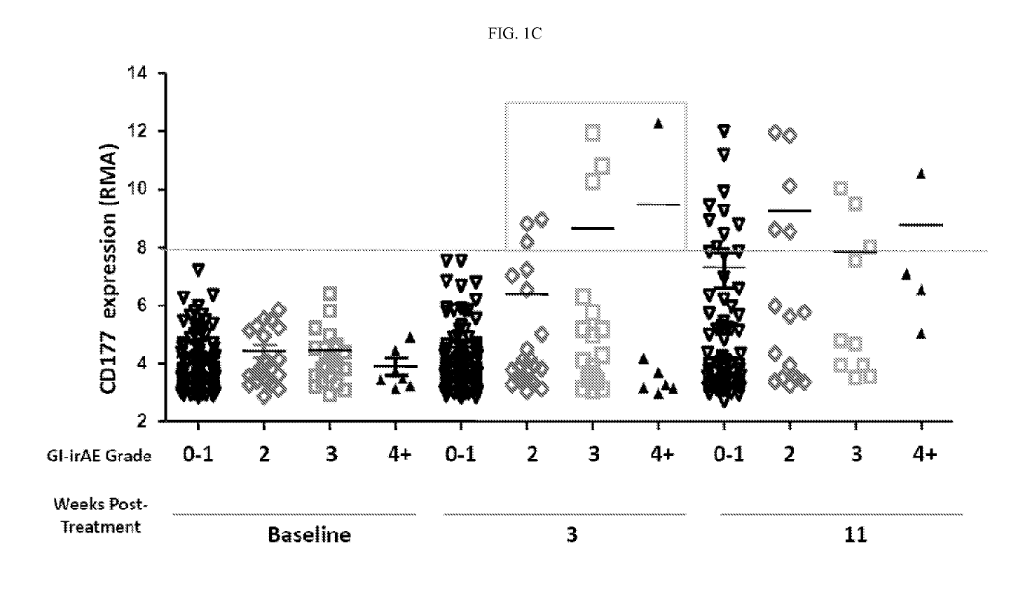

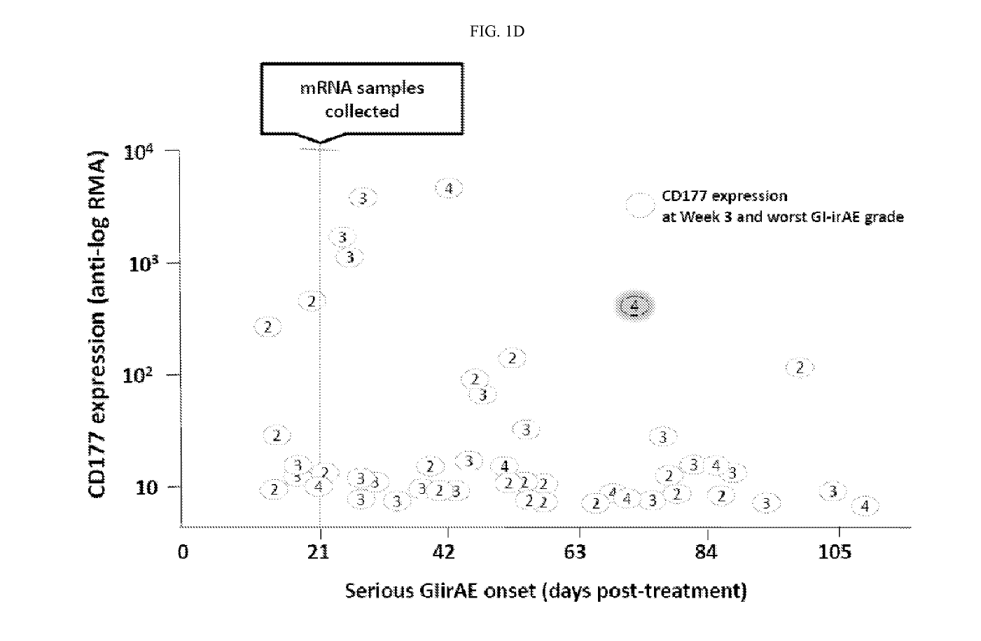

Gene Expression Profiles of Post-Treatment Samples. To identify early on-treatment predictors of ipilimumab-associated GI-irAEs, post-baseline expression levels of the 9697 non-control probe sets were compared between the GI-irAE and No-GI-irAE groups. Thirty five and 47 probe sets were identified to have a mean expression ratio of at least 1.5 for the week 3 and 11 samples, respectively, and a P value ≦0.05 (FDR=0.50) for the hypothesis test comparing the GI and No-GI-irAE groups. Since most ipilimumab-associated GI-irAEs occur after the second or third dose of ipilimumab, the 35 probe sets differentially expressed at week 3 are of particular interest, as they might serve as early predictors to help identify patients who experience GI-irAEs after the second dose, given at week 3 (Table 1B). The probe set that exhibited the largest differential expression corresponds to the neutrophil specific marker, CD177, a glycosylphosphatidylinositol (GPI)-linked cell surface molecule (Lalezari, P. et al., J. Clin. Invest., 50:1108-1115 (1971)). Although CD177 showed no significant differential expression in the baseline samples, its mean expression in the GI-irAE group was 12.2 fold higher than that of the No-GI-irAE group at week 3 (P=7.57E-03). In addition, CD177 showed significant increase in mean expression from baseline to week 3 in the GI-irAE group but not in the No-GI-irAE patients (P=9.78E-04) (Table 2). Importantly, the mean increase in the CD177 expression was not associated with the clinical activity (CA) of ipilimumab (P=2.28E-01) (FIG. 1A). Expression levels of CD177 in these samples were confirmed by quantitative PCR, showing statistically significant differences between the GI-irAE and No-GI-irAE groups (unadjusted P<0.005) or changes over time (unadjusted P<0.0001). These data suggested that CD177 was not only a potential early predictor of GI-irAE but its increase might also be a consequence of the treatment with ipilimumab independently from the its clinical activity.

Evaluation of CD177 as an Early Predictor of GI-irAEs. Since CD177 is a neutrophil surface marker, the inventors examined the relationship between APBNC and the expression levels of CD177. APBNC were measured for all patients in these two clinical studies at various time points as a component of the standard hematology panel. The mean APBNCs increased gradually with the ipilimumab treatment regardless of the GI-irAE grades, with the largest increases occurring between weeks 9 and 11 (FIG. 1B). APBNCs also correlated with the grades of GI-irAE, where patients having larger numbers of APBNCs experienced more severe GI-irAEs. Interestingly, the increase in mean expression of CD177 preceded any significant increase in APBNC, suggesting that the CD177 expression might be a more sensitive early predictor of the GI-irAEs associated with the ipilimumab treatment.

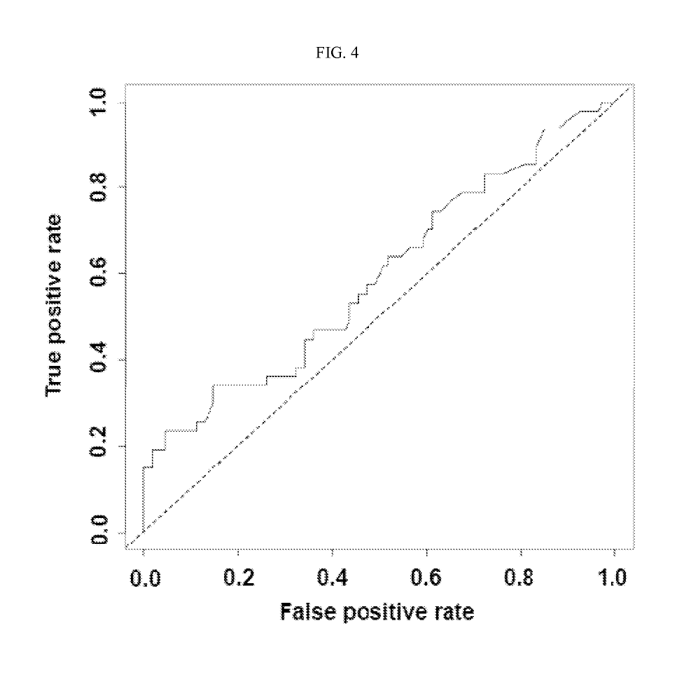

The expression of CD177 at week 3 showed a wide inter-individual variability with considerably higher values in a few patients than the rest (FIG. 1C). However, strikingly all seven patients with high levels of CD177 expression (RMA 8) at week 3 reported serious GI-irAEs (G2+) during the course of the treatment, suggesting high specificity of this biomarker above this threshold. However, many patients with grade 2+GI-irAEs had a normalized expression level <8 for CD177 at week 3, suggesting low sensitivity of the biomarker in predicting GI-irAEs (FIG. 4). At Week 11 post-treatment, as high expression levels of CD177 were found in both the GI-irAE and No-GI-irAE groups. It had to be noted that many patients who reported serious adverse events had already discontinued the treatment before this time point, so the data from week 11 might be biased.

We also explored the association of CD177 expression and the onset of GI-irAE in individual patients to establish the value of CD177 expression as an early predictor. Out of 44 reported cases of serious GI-irAE (G2+) in CA184004 and 007 studies, with matching mRNA data at week 3, six patients reported the first serious incident before or on day 21±3 days (the nominal date when the mRNA sample was collected), whereas the other 38 cases were reported after this date (FIG. 1D). The four highest values of the CD177 expression belonged to patients who reported the first serious GI-irAE between days 26 (2-5 days after mRNA collection) and 43 after receiving the first dose of ipilimumab. Of note, one of the patients who showed high CD177 expression levels at week 3, reported a G2 GI-irAE on day 72 (FIG. 1D, marked in gray shade circle), but progressed to a Grade 4 which ultimately led to a fatal GI perforation in this patient. These data suggest that although considerable increase in CD177 gene expression is closely associated with the onset of GI-irAE but early increases might also serve to predict GI-irAEs that might develop at much later time points. However, because CD177 had low sensitivity, this biomarker may not be able to identify most future GI-irAEs. Analysis of additional patient cohorts is warranted to further refine the overall sensitivity of this marker in order to support the observed GI-irAE correlation.

Association of Other Neutrophil Associated Genes and CD177. CD177 is a glycoprotein expressed exclusively by neutrophils, neutrophilic metamyelocytes, and myelocytes, but not by any other blood cells (Stroncek, D. F. et al., Transfusion, 36:168-174 (1996); Stroncek, D. F. et al., Blood, 75:744-755 (1990)). Therefore the inventors specifically searched for neutrophil-associated probsets, to better understand the implication of this granulocyte subtype as an early predictor of GI-irAEs. These included genes encoding for granule-associated proteins such as olfactomedin 4 (OLFM4) (Rosenbauer, F. et al., Blood, 103:4294-4301 (2004)), azurocidin 1 (AZU1), lactoferrin (LTF) (Miyauchi, J. et al., Cell Tissue Res., 247:249-258 (1987)), cathelicidin (CAMP), myeloperoxidase (MPO) (Falloon, J. et al., J. Allergy Clin. Immunol., 77:653-662 (1986)), bactericidal/permeability increasing protein (BPI), Defensins (DEFA4) (Gabay, J. E. et al., Proc. Natl. Acad. Sci. USA, 86:5610-5614 (1989)), the neutrophil elastase (ELANE) (Korkmaz, B. et al., Pharmacol. Rev., 62:726-759 (2010)), cathepsin G (CTSG) (Korkmaz, B. et al., Pharmacol. Rev., 62:726-759 (2010)), CEACAM-6, CEACAM-8 (Zhao, L. et al., Br. J. Haematol., 125:666-673 (2004)) and other non-granule, neutrophil-associated genes such as CEACAM1. The mRNA GENECHIP® array HT-HG-U133A 96-array included many of these probsets. Although an apparent greater expression of each of these genes was found in those samples with high levels of CD177 expression (FIG. 2A), only the expression of CEACAM1 was significantly linearly correlated with that of CD177 at week 3 (r=0.75, P=7.2E-30 between 219669_at, probe set for CD177, and 206576_s_at, probe set for CEACAM1). Consequently, the pattern of mean gene expression of CEACAM1 over time was similar to that for CD177 (FIGS. 1A and 2B).

Pathway Analysis of Changes in Gene Expression from Baseline to Week 11. Fifty-eight and 247 probe sets were identified as having at least a 1.5-fold change from baseline in week 3 and 11 samples, respectively, and P≦0.05 (FDR=0.055) for the test of a time effect on expression (Tables 2A and 2B). The inventors performed a pathway analysis using the IPA software on the 247 differentially expressed probe sets since the size of this gene set was amenable to such analysis. The top biological processes that exhibited changes during ipilimumab treatment included pathways of cell proliferation and metabolism, and immune-related pathways such as IL-10 signaling, IL-8 signaling, and B cell development (data not shown).

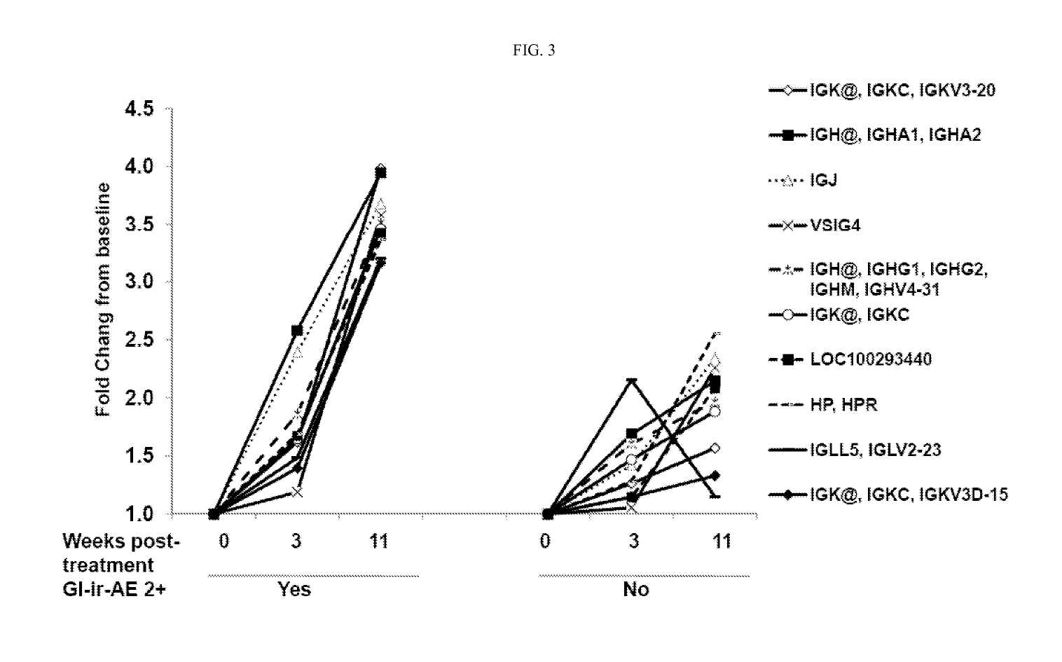

Increase in Immunoglobulin-Related Genes 11 Weeks Post-Treatment. While CD177 exhibited the largest change from baseline at both time points, immunoglobulin-related genes dominated both lists, including IGHA1, IGHA2, IGHG1, and IGHV4-31, all of which showed significant increases in expression 11 weeks after baseline. Slight increases in expression of some of these genes were already apparent in week 3 samples, suggesting an early onset of antibody response with much larger expansion at later time points (Table 2 and FIG. 3). Notably, increases in expression of these genes over time were more prominent in the GI-irAE groups than in the No-GI-irAE group. There was no corresponding increase in B-cell marker genes (such as CD20), suggesting that the cell types responsible for the increased expression of immunoglobulin genes may not have been B-cells, but later-stage B-lineage cells such as plasma cells.

Confirmation of Findings in an Independent Study (CA184078). Whole blood samples from the clinical study CA184078 were independently analyzed using the same statistical model. In this study, 20 patients were treated with ipilimumab monotherapy at 10 mg/kg every 3 weeks for 4 doses. The mean CD177 expression ratio comparing GI-irAE and No-GI-irAE groups at week 3 and 11 was 4.3 and 12.0, respectively, with no significant difference at baseline. In the GI-irAE group, the mean fold change from baseline to week 3 and 11 was 4.8 and 15.3, respectively. By contrast, in the No-GI-irAE group, these changes were negligible (1.1 and 1.2, respectively). Expression changes similar to those seen in the other two studies were observed for CEACAM1 and most of the granule-associated genes, with significant changes from baseline to week 11 in the GI-irAE group but not in the No-GI-irAE group (Table 5).

### DISCUSSION

The goal of melanoma treatment with ipilimumab is to achieve complete or partial tumor regression, prolong survival and improve quality of life. However, although only a proportion of patients may benefit from the treatment, all patients are exposed to the potential toxicities associated with this agent. Gastrointestinal irAEs such as diarrhea and colitis are one of the most common and sometime serious forms of adverse events associated with ipilimumab. Identification of biomarkers that can predict their incidence might assist in enhanced management of these drug related toxicities and improve patient care. The present retrospective gene expression analysis was undertaken to identify this class of biomarkers in peripheral blood of patients treated with ipilimumab. In a previous report from the CA184007 trial in which colonic biopsies were collected, changes in the colonic mucosa following onset of diarrhea appeared more severe than those observed in the prespecified biopsies following the first dose of ipilimumab. Histopathologic examination of the biopsies revealed active colitis with marked neutrophilic infiltration into lamina propria to be the most striking characteristic of the affected tissue. In these biopsies, foci of neutrophilic cryptitis, crypt abscesses, glandular destruction, and erosions of the mucosal surface were also apparent early after the start of the treatment (Minor, D. R. et al., Cancer Biother. Radiopharm., 24:321-325 (2009)). However, collection of colonic biopsies is considered an invasive procedure and therefore, peripheral blood surrogate markers are preferred.

The GI-irAEs onset is most commonly observed after the second or third doses of ipilimumab. The inventors found changes in the peripheral gene expression profile of patients who experienced serious GI-irAE, that were apparent only 3 weeks post-ipilimumab treatment. The greatest differential gene set included a number of neutrophil-associated genes. In particular significantly greater mean expression of the neutrophil surface marker CD177 was detected at this early time point in a subset of patients who experienced GI-irAEs. Neutrophil granulocytes are the most abundant type of white blood cells in mammals and form an essential part of the innate immune system. During the acute phase of inflammation, particularly as a result of bacterial infection, environmental exposure (Jacobs, L. et al., Environ. Health, 9:64 (2010)), and some cancers (De Larco, J. E. et al., Clin. Cancer Res., 10:4895-4900 (2004); Waugh, D. J. et al., Clin. Cancer Res., 14:6735-6741 (2008)), neutrophils are of the first-responders of inflammatory cells to migrate towards the site of inflammation. They migrate through the blood vessels, then through interstitial tissue, following chemical signals such as Interleukin-8 (IL-8), C5a, and Leukotriene B4 in a process called chemotaxis (Follin, P. et al., Eur. J. Haematol., 47:71-76 (1991)). CD177 is unique marker for neutrophils (Stroncek, D. F. et al., Transfusion, 36:168-174 (1996); Stroncek, D. F. et al., Blood, 75:744-755 (1990)) and is upregulated upon neutrophil activation during acute inflammatory responses toward stimuli such as bacterial infections (Gohring, K. et al., Br. J. Haematol., 126:252-254 (2004)). Significant increase in the mean expression of this marker was detected in patients who experienced serious GI-irAE, independently from their clinical response to ipilimumab. Indeed, the top seven patients who had the highest expression level of CD177 (above RMA 8) were those who already had or would experience GI-irAE within a few days to three weeks after that time point (when the 3 week mRNA sample was collected). Currently ipilimumab GI toxicity is safely managed by following clear guidelines using a specific immunosuppressive regimen. CA184004 was one of the earlier ipilimumab monotherapy trials, where these guidelines were still not in place. In that trial, two patients died of serious GI-irAE and intestinal perforation. One of these patients had the last dose of ipilimumab on Day 22, reported Grade 3 diarrhea on Day 42 and died on Day 59 from Grade 5 large intestine perforation. This patient did not provide a mRNA sample at the 3 week time point. A second patient had the last dose of ipilimumab on Day 43 and died on Day 142. This patient reported prolonged rash, pruritis and prolonged Grade 1-2 diarrhea starting with the first episode of Grade 2 on day 72 which developed to Grade 4 large intestine perforation on Day 138. This patient was treated with pulse steroids but did not receive high-dose steroids with 1 month taper. The WB mRNA gene expression profile from this patient showed high CD177 expression with 42.5 fold increase from baseline to week 3, almost 50 days before the onset of the first GI-irAE episode, suggesting the value of CD177 gene expression as an early predictor of the severe GI-irAE.

Another neutrophil marker CEACAM1 also showed consistent increase in mean expression from baseline to post-induction phase with the greatest increase in the GI-irAE group. Whereas CD177 is mostly an activation marker for neutrophils, CEACAM1 mediates adherence of activated neutrophils to cytokine-activated endothelium (Skubitz, K. M. et al., Biopolymers, 96:25-31 (2011)) and has been suggested to play a role in immune-mediated diseases of the intestine. CEACAM1 expression has been reported in T cells of the lamina propria of small intestine in patients with celiac disease and in the large intestine of those with inflammatory bowel disease (IBD) (Costello, C. M. et al., PLoS Med., 2:e199 (2005)). CD177 has also been shown to recognize an endothelial cell junction molecule, PECAM-1 which contributes to interactions between neutrophils and endothelial cells, mediating trans-endothelial migration in the context of inflammatory cell recruitment (Sachs, U. J. et al., J. Biol. Chem., 282:23603-23612 (2007)).

These findings suggested an apparent association between neutrophils, CD177 and ipilimumab associated GI-irAEs and led us to search for other neutrophil-associated genes in the microarray data. Degranulation is the process by which neutrophils release an assortment of proteins (Witko-Sarsat, V. et al., Lab. Invest., 80:617-653 (2000)) such as such as lactoferrin (LFT) (Miyauchi, J. et al., Cell Tissue Res., 247:249-258 (1987)), cathelicidin (CAMP), myeloperoxidase (MPO) (Falloon, J. et al., J. Allergy Clin. Immunol., 77:653-662 (1986)), bactericidal/permeability increasing protein (BPI), Defensins (DEFA4) (Gabay, J. E. et al., Proc. Natl. Acad. Sci. USA, 86:5610-5614 (1989)), elastase (ELANE) (Korkmaz, B. et al., Pharmacol. Rev., 62:726-759 (2010)) and cathepsin G (CTSG) (Korkmaz, B. et al., Pharmacol. Rev., 62:726-759 (2010)) into the extracellular space. In alignment with CD177, the expression of all of these genes was also greater in those samples with high levels of CD177 expression, confirming an important role of neutrophil activation and function in mediating severe GI-irAEs. These observations were also confirmed in another data set from an independent ipilimumab clinical trial, CA184078, in which higher mean expression of CD177 and CEACAM1 were found in the GI-irAE group.

Interestingly our list of early predictors of GI-irAE shared a number of common elements with those reported to confer resistance to intravenous corticosteroid therapy in children with ulcerative colitis (Kabakchiev, B. et al., PLoS One, 5(9):e13085 (2010)). Some of identified genes shared between the two studies were: CD177, CEACAM1, OLFM4, MMP8, BPI, CLC, HP and LCN2. In that study, post-treatment samples were collected only three days after the start of the corticosteroid treatment, still significant differences between the expressions of these genes were detected between resistant and responder patients. In our study, the earliest post-treatment blood samples were collected 3 weeks after the first dose of ipilimumab while the major changes in the gene expression occurred within this time period, suggesting that it might be possible to detect this predictive profile even at an earlier time within this period. In any event, these changes preceded the significant changes in the PBNC, which did not occur within the first 3 weeks from the start of the treatment.

More importantly, CD177 expression did not show any apparent association with clinical response to ipilimumab, suggesting that the GI-irAE (and thereof neutrophil activation) is most likely independent of response, or that its inhibition or blockade might not affect the clinical response. Indeed, association of expression levels of CD177 and clinical response was somewhat reversed, with patients showing no clinical activity displaying higher post-treatment mean expression of CD177. This is not surprising, as together with T regulatory and M2 macrophages, high intratumoral neutrophils have been suspected to stimulate cancer growth (Zitvogel, L. et al., Cancer Res., 70:9538-9543 (2010)).

Another interesting finding in our analysis was the considerable increase in the number and expression of immunoglobulin-related genes at 11 weeks post-treatment in patients who had GI-irAEs. In normal healthy people, the humoral response to enteric flora is maintained in homeostasis. Dysregulation of this homeostasis, manifested as increasing antibody levels to select enteric flora, is characteristic of gastrointestinal disorders such as IBD but not acute GI inflammation (i.e., diverticulitis/infection) (Minor, D. R. et al., Cancer Biother. Radiopharm., 24:321-325 (2009); Hamid, O. et al., AJ Clin. Oncol., 27(Suppl.), Abstract 9008 (2009); Lalezari, P. et al., J. Clin. Invest., 50:1108-1115 (1971)). In a previous report from the CA184007 trial, ipilimumab was found to induce antibody responses to select enteric flora such as Pseudomonas anti-I2, Saccharomyces cerevisiae antibody (ASCA), or CBir flagellin antibody (CBir1). However, no strong associations between a positive level of antibody responses toward these specific bacteria and GI-irAEs were observed. Although gene expression analysis does not provide information of the specificity of the induced antibodies, it still indicates that the immunoglobulin production machinery has been turned on in patients experiencing GI-irAE. In the absence of infections by external pathogens, this response could most probably be due to an autoimmune reaction toward internal stimuli that might be as well the intestinal flora.

An additional analysis of the data obtained for CD177 and CEACAM1 was performed in order to assess whether establishing a baseline level of expression for these two genes across a patient population at week 1 compared to establishing a baseline level of expression for these two genes from an individual patient at week 1 was better at predicting the incidence of GI-irAE (see Table 3). The results demonstrate that using an individual patient's expression level at week 1 resulted in a modest increase in specificity in predicting the incidence of a GI-irAE when either CD177 or CEAMCAM1 was measured, particularly when the expression level at week 4 was high, although there was a slight decrease in the sensitivity of the assay.

In conclusion, the inventors have identified early changes in gene expression in patients treated with ipilimumab that in some patients might predict the incidence of later GI-irAEs. These gene expression changes, together with prior histopathologic examination of the affected tissue, point to an important role of neutrophils in the onset of GI-irAEs in these patients. High expression of CD177 at week 3 was a very specific biomarker for grade 2+ GI-irAEs, as all patients who had no such event displayed expression levels below a certain threshold (normalized expression level=8). However, because of its low sensitivity as a biomarker, the current data suggests CD177 expression alone may not be entirely determinative for predicting which patients will develop GI-irAEs because the data do not entirely preclude the possibility GI-irAEs may occur in patients with low CD177 expression. The earliest on-treatment sample collection was 3 weeks after first ipilimumab dose. Therefore it is not clear how much increases in CD177 expression preceded the onset of GI-irAEs. This study identified potential biomarkers of ipilimumab toxicity that have biological plausibility. Additional validation of the listed biomarkers may be pursued in a larger controlled phase III trial.

TABLE 1A
Predictive Genes

| | | | Fold difference between | | |
|---|---|---|---|---|---|
| | | | GlirAE vs. no-GlirAE | | |
| Probeset | Gene | P value | Baseline | Week 3 | Week 11 |
| 205456_at | CD3E | 5.1E−03 | 2.4 | 2.4 | 2.0 |
| 208611_s_at | SPTAN1 | 1.6E−02 | 2.2 | 2.3 | 2.0 |
| 200707_at | PRKCSH | 4.2E−02 | 2.1 | 2.0 | 1.5 |
| 204116_at | IL2RG | 3.0E−02 | 2.0 | 2.1 | 1.7 |
| 210125_s_at | BANF1 | 5.9E−03 | 1.9 | 2.0 | 1.7 |
| 215236_s_at | PICALM | 2.4E−02 | 1.8 | 1.7 | 1.6 |
| 209131_s_at | SNAP23 | 6.1E−03 | 1.8 | 1.7 | 1.5 |
| 204192_at | CD37 | 1.7E−02 | 1.8 | 1.9 | 1.6 |
| 214080_x_at | PRKCSH | 9.8E−03 | 1.7 | 1.6 | 1.4 |
| 200041_s_at | BAT1 | 1.8E−02 | 1.7 | 1.8 | 1.6 |
| 210935_s_at | WDR1 | 1.2E−02 | 1.7 | 1.6 | 1.6 |
| 210023_s_at | PCGF1 | 1.8E−02 | 1.7 | 1.7 | 1.6 |
| 205513_at | TCN1 | 2.2E−02 | 1.7 | 1.1 | 1.9 |
| 203547_at | CD4 | 2.0E−02 | 1.7 | 1.7 | 1.5 |
| 211825_s_at | EWSR1/// | 5.7E−03 | 1.6 | 1.5 | 1.5 |
| | FLI1 | | | | |
| 211272_s_at | DGKA | 4.8E−03 | 1.6 | 1.7 | 1.6 |
| 207419_s_at | RAC2 | 4.1E−03 | 1.6 | 1.6 | 1.6 |
| 213048_s_at | — | 2.2E−02 | 1.6 | 1.6 | 1.4 |
| 212193_s_at | LARP1 | 2.5E−02 | 1.6 | 1.6 | 1.4 |
| 201337_s_at | VAMP3 | 3.4E−02 | 1.6 | 1.2 | 1.4 |
| 211413_s_at | PADI4 | 8.3E−03 | 1.6 | 1.9 | 1.9 |
| 212595_s_at | DAZAP2 | 3.0E−02 | 1.6 | 1.3 | 1.3 |
| 201367_s_at | ZFP36L2 | 1.3E−02 | 1.5 | 1.4 | 1.3 |
| 202101_s_at | RALB | 6.4E−03 | 1.5 | 1.5 | 1.3 |
| 214544_s_at | SNAP23 | 2.5E−02 | 1.5 | 1.3 | 1.3 |
| 203828_s_at | IL32 | 2.0E−02 | 1.5 | 1.6 | 1.5 |
| 203385_at | DGKA | 7.6E−03 | 1.5 | 1.6 | 1.5 |

TABLE 1B
Early Predictive Genes

| | | | Fold difference | | | | | | |
|---|---|---|---|---|---|---|---|---|---|
| | | | between GlirAE vs. | | | Fold change in | | Fold change in No- | |
| | | | no-GlirAE | | | GlirAE by time | | GlirAE by time | |
| Probeset | Gene | P value | 1 | 3 | 11 | W 0 vs. 3 | W 0 vs. 11 | W 0 vs. 3 | W 0 vs. 11 |
| 219669_at | CD177 | 7.6E−03 | 1.1 | 12.2 | 2.9 | 14.5 | 22.2 | 1.3 | 8.6 |
| 205456_at | CD3E | 5.1E−03 | 2.4 | 2.4 | 2.0 | 1.1 | −1.0 | 1.1 | 1.2 |
| 208611_s_at | SPTAN1 | 1.6E−02 | 2.2 | 2.3 | 2.0 | 1.3 | 1.2 | 1.3 | 1.3 |
| 204116_at | IL2RG | 3.0E−02 | 2.0 | 2.1 | 1.7 | 1.2 | −1.0 | 1.1 | 1.2 |
| 200707_at | PRKCSH | 4.2E−02 | 2.1 | 2.0 | 1.5 | 1.1 | −1.2 | 1.1 | 1.2 |
| 210125_s_at | BANF1 | 5.9E−03 | 1.9 | 2.0 | 1.7 | 1.1 | −1.1 | 1.1 | 1.0 |
| 204192_at | CD37 | 1.7E−02 | 1.8 | 1.9 | 1.6 | 1.0 | −1.1 | −1.1 | −1.0 |
| 211413_s_at | PADI4 | 8.3E−03 | 1.6 | 1.9 | 1.9 | 1.1 | 1.3 | −1.1 | 1.1 |
| 215783_s_at | ALPL | 8.3E−03 | 1.1 | 1.8 | 1.6 | 1.6 | 2.0 | −1.0 | 1.4 |
| 207384_at | PGLYRP1 | 2.5E−02 | 1.3 | 1.8 | 1.6 | 1.4 | 1.9 | 1.1 | 1.5 |
| 200041_s_at | BAT1 | 1.8E−02 | 1.7 | 1.8 | 1.6 | 1.1 | −1.0 | 1.0 | 1.1 |
| 211272_s_at | DGKA | 4.8E−03 | 1.6 | 1.7 | 1.6 | 1.2 | 1.2 | 1.1 | 1.2 |
| 203547_at | CD4 | 2.0E−02 | 1.7 | 1.7 | 1.5 | 1.1 | −1.2 | 1.0 | −1.0 |
| 210023_s_at | PCGF1 | 1.8E−02 | 1.7 | 1.7 | 1.6 | 1.2 | 1.1 | 1.2 | 1.2 |
| 204480_s_at | C9orf16 | 3.2E−02 | 1.4 | 1.7 | 1.2 | 1.2 | −1.1 | −1.1 | 1.1 |
| 215236_s_at | PICALM | 2.4E−02 | 1.8 | 1.7 | 1.6 | −1.1 | −1.2 | −1.0 | −1.0 |
| 209131_s_at | SNAP23 | 6.1E−03 | 1.8 | 1.7 | 1.5 | −1.0 | −1.1 | 1.1 | 1.1 |
| 202118_s_at | CPNE3 | 1.3E−03 | 1.5 | 1.7 | 1.6 | 1.1 | 1.1 | −1.1 | 1.1 |
| 212193_s_at | LARP1 | 2.5E−02 | 1.6 | 1.6 | 1.4 | 1.2 | 1.1 | 1.1 | 1.2 |
| 203828_s_at | IL32 | 2.0E−02 | 1.5 | 1.6 | 1.5 | 1.3 | 1.2 | 1.3 | 1.2 |
| 202742_s_at | PRKACB | 1.1E−02 | 1.3 | 1.6 | 1.3 | 1.3 | 1.1 | 1.0 | 1.1 |
| 214080_x_at | PRKCSH | 9.8E−03 | 1.7 | 1.6 | 1.4 | 1.0 | −1.2 | 1.1 | 1.0 |
| 213048_s_at | — | 2.2E−02 | 1.6 | 1.6 | 1.4 | 1.1 | −1.0 | 1.1 | 1.1 |
| 210935_s_at | WDR1 | 1.2E−02 | 1.7 | 1.6 | 1.6 | 1.0 | −1.0 | 1.1 | 1.0 |
| 207419_s_at | RAC2 | 4.1E−03 | 1.6 | 1.6 | 1.6 | 1.1 | 1.1 | 1.1 | 1.1 |
| 214383_x_at | KLHDC3 | 2.4E−02 | 1.4 | 1.6 | 1.3 | 1.1 | −1.1 | 1.0 | 1.0 |
| 207691_x_at | ENTPD1 | 3.7E−02 | 1.3 | 1.6 | 1.3 | 1.2 | 1.2 | 1.0 | 1.2 |

TABLE 2A
Genes Selected Based on Change from Baseline to Week 3

| | | | Mean fold change from | | Mean fold change from BL | |
|---|---|---|---|---|---|---|
| | | | BL in GI-irAE group | | in No-GI-irAE group | |
| Probe Set | Gene | P Value | BL vs. W 3 | BL vs. W 11 | BL vs. W 3 | BL vs. W 11 |
| 219669_at | CD177 | 9.8E−04 | 14.5 | 22.2 | 1.3 | 8.6 |
| 209773_s_at | RRM2 | 2.8E−11 | 3.1 | 5.9 | 2.8 | 2.8 |
| 201893_x_at | DCN | 1.9E−02 | 2.7 | −1.0 | −1.0 | −1.0 |
| 215076_s_at | COL3A1 | 1.4E−02 | 2.6 | −1.0 | −1.0 | −1.0 |
| 217022_s_at | IGH@/IGHA1/IGHA2/LOC100126583 | 2.9E−11 | 2.6 | 3.9 | 1.7 | 2.1 |
| 206513_at | AIM2 | 4.6E−06 | 2.4 | 2.8 | 1.5 | 2.0 |
| 212592_at | IGJ | 1.4E−04 | 2.4 | 3.7 | 1.4 | 2.3 |
| 216950_s_at | FCGR1A/FCGR1C | 3.7E−06 | 2.1 | 2.2 | 1.9 | 1.9 |
| 214511_x_at | FCGR1B | 7.4E−07 | 2.1 | 2.2 | 1.8 | 1.9 |
| 206207_at | CLC | 3.5E−10 | 2.0 | 1.1 | 1.8 | 1.5 |
| 203554_x_at | PTTG1 | 2.6E−14 | 2.0 | 3.0 | 1.8 | 1.8 |
| 202503_s_at | KIAA0101 | 3.0E−05 | 1.9 | 2.6 | 1.4 | 1.6 |
| 211430_s_at | IGH@/IGHG1/IGHG2/IGHM/IGHV4-31/ | 2.0E−06 | 1.9 | 3.5 | 1.6 | 2.0 |
| | LOC100290146/LOC100294459 | | | | | |
| 204170_s_at | CKS2 | 1.1E−02 | 1.8 | 1.5 | 1.1 | 1.2 |
| 202589_at | TYMS | 4.8E−08 | 1.8 | 2.7 | 1.8 | 1.9 |
| 209138_x_at | IGL@ | 2.0E−08 | 1.7 | 2.9 | 1.4 | 1.7 |
| 209348_s_at | MAF | 5.0E−04 | 1.7 | 1.5 | 1.3 | 1.4 |
| 206576_s_at | CEACAM1 | 2.4E−04 | 1.7 | 2.8 | 1.3 | 1.5 |
| 208470_s_at | HP/HPR | 7.3E−04 | 1.7 | 3.4 | 1.3 | 2.6 |
| 214677_x_at | CYAT1/IGLV1-44 | 6.6E−10 | 1.7 | 2.5 | 1.3 | 1.6 |
| 205844_at | VNN1 | 5.2E−04 | 1.7 | 3.3 | −1.3 | 1.8 |
| 217148_x_at | LOC100293440 | 1.5E−05 | 1.7 | 3.4 | 1.1 | 2.1 |
| 202269_x_at | GBP1 | 8.2E−07 | 1.7 | 1.6 | 1.7 | 1.5 |
| 215176_x_at | IGK@/IGKC/LOC100291464 | 4.9E−07 | 1.6 | 3.5 | 1.5 | 1.9 |
| 200986_at | SERPING1 | 8.5E−03 | 1.6 | 1.5 | 2.1 | 1.7 |
| 209369_at | ANXA3 | 3.2E−06 | 1.6 | 2.5 | 1.2 | 1.9 |
| 211645_x_at | — | 2.8E−05 | 1.6 | 3.8 | 1.3 | 1.7 |
| 215783_s_at | ALPL | 2.6E−07 | 1.6 | 2.0 | −1.0 | 1.4 |
| 211644_x_at | IGK@/IGKC/IGKV3-20/LOC100291682 | 5.2E−05 | 1.6 | 4.0 | 1.3 | 1.6 |
| 201754_at | COX6C | 1.7E−02 | 1.6 | 1.3 | 1.1 | 1.2 |
| 218728_s_at | CNIH4 | 9.6E−03 | 1.6 | 1.4 | 1.0 | 1.3 |
| 201761_at | MTHFD2 | 1.3E−02 | 1.6 | 1.4 | 1.2 | 1.4 |
| 202270_at | GBP1 | 7.5E−04 | 1.6 | 1.7 | 1.6 | 1.4 |
| 202870_s_at | CDC20 | 6.2E−11 | 1.6 | 2.7 | 1.6 | 1.7 |
| 219978_s_at | NUSAP1 | 1.2E−10 | 1.6 | 2.4 | 1.6 | 1.6 |
| 215379_x_at | IGLV1-44 | 5.4E−10 | 1.6 | 2.3 | 1.3 | 1.5 |
| 214669_x_at | IGKC | 1.2E−10 | 1.6 | 2.5 | 1.3 | 1.6 |
| 207111_at | EMR1 | 1.1E−04 | 1.6 | 1.2 | 1.3 | 1.2 |
| 219684_at | RTP4 | 1.2E−04 | 1.6 | 1.5 | 1.4 | 1.3 |
| 202746_at | ITM2A | 4.0E−04 | 1.5 | 1.6 | 1.3 | 1.4 |
| 210052_s_at | TPX2 | 1.9E−08 | 1.5 | 2.0 | 1.5 | 1.5 |
| 215121_x_at | CYAT1/IGLV1-44 | 1.7E−09 | 1.5 | 2.1 | 1.3 | 1.5 |
| 201294_s_at | WSB1 | 1.8E−03 | 1.5 | 1.3 | 1.1 | 1.3 |
| 221651_x_at | IGK@/IGKC | 3.5E−11 | 1.5 | 2.0 | 1.3 | 1.4 |
| 221671_x_at | IGK@/IGKC | 1.8E−11 | 1.5 | 2.0 | 1.3 | 1.4 |
| 211889_x_at | CEACAM1 | 1.2E−04 | 1.5 | 2.4 | 1.3 | 1.5 |
| 216207_x_at | IGKV1D-13 | 5.1E−06 | 1.5 | 2.3 | 1.2 | 1.5 |
| 218447_at | C16orf61 | 1.1E−04 | 1.5 | 1.4 | 1.1 | 1.2 |
| 209395_at | CHI3L1 | 1.1E−05 | −1.5 | −1.6 | −1.2 | −1.2 |
| 209396_s_at | CHI3L1 | 6.6E−06 | −1.5 | −1.6 | −1.2 | −1.2 |
| 211657_at | CEACAM6 | 4.8E−04 | −1.6 | 1.4 | −1.0 | 1.5 |
| 203948_s_at | MPO | 4.9E−04 | −1.6 | −1.0 | 1.0 | 1.6 |
| 207269_at | DEFA4 | 4.8E−04 | −1.6 | 1.5 | 1.0 | 1.7 |
| 203949_at | MPO | 9.8E−05 | −1.6 | 1.1 | 1.0 | 1.4 |
| 206871_at | ELANE | 2.7E−04 | −1.8 | 1.4 | 1.1 | 1.9 |
| 212768_s_at | OLFM4 | 6.6E−04 | −1.8 | 4.0 | 1.2 | 3.6 |
| 203757_s_at | CEACAM6 | 1.2E−03 | −1.8 | 1.4 | 1.0 | 1.6 |
| 206676_at | CEACAM8 | 3.7E−04 | −1.9 | 1.9 | 1.1 | 2.0 |

TABLE 2B
Genes Selected Based on Change from Baseline to Week 11

| | | | Mean fold change from | | Mean fold change from | |
|---|---|---|---|---|---|---|
| | | | BL in GI-irAE group | | BL in No-GI-irAE group | |
| Probe Set | Gene | P Value | BL vs. W 3 | BL vs. W 11 | BL vs. W 3 | BL vs. W 11 |
| 219669_at | CD177 | 9.8E−04 | 14.5 | 22.2 | 1.3 | 8.6 |
| 209773_s_at | RRM2 | 2.8E−11 | 3.1 | 5.9 | 2.8 | 2.8 |
| 211644_x_at | IGK@/IGKC/IGKV3-20/LOC100291682 | 5.2E−05 | 1.6 | 4.0 | 1.3 | 1.6 |
| 212768_s_at | OLFM4 | 6.6E−04 | −1.8 | 4.0 | 1.2 | 3.6 |
| 217022_s_at | IGH@/IGHA1/IGHA2/LOC100126583 | 2.9E−11 | 2.6 | 3.9 | 1.7 | 2.1 |
| 211645_x_at | — | 2.8E−05 | 1.6 | 3.8 | 1.3 | 1.7 |
| 212592_at | IGJ | 1.4E−04 | 2.4 | 3.7 | 1.4 | 2.3 |
| 204787_at | VSIG4 | 2.8E−04 | 1.2 | 3.6 | 1.1 | 2.3 |
| 211430_s_at | IGH@/IGHG1/IGHG2/IGHM/IGHV4-31/ | 2.0E−06 | 1.9 | 3.5 | 1.6 | 2.0 |
| 214768_x_at | — | 3.1E−04 | 1.5 | 3.5 | 1.2 | 1.3 |
| 215176_x_at | IGK@/IGKC/LOC100291464 | 4.9E−07 | 1.6 | 3.5 | 1.5 | 1.9 |
| 217148_x_at | LOC100293440 | 1.5E−05 | 1.7 | 3.4 | 1.1 | 2.1 |
| 208470_s_at | HP/HPR | 7.3E−04 | 1.7 | 3.4 | 1.3 | 2.6 |
| 205844_at | VNN1 | 5.2E−04 | 1.7 | 3.3 | −1.3 | 1.8 |
| 217235_x_at | IGLL5/IGLV2-23 | 2.1E−05 | 1.5 | 3.2 | 1.1 | 1.7 |
| 210999_s_at | GRB10 | 5.2E−04 | 1.3 | 3.2 | −1.1 | 2.4 |
| 212793_at | DAAM2 | 2.7E−02 | −1.1 | 3.2 | −1.4 | 1.9 |
| 206618_at | IL18R1 | 7.6E−04 | 1.3 | 3.2 | −1.1 | 2.1 |
| 211643_x_at | IGK@/IGKC/IGKV3D-15 | 1.0E−04 | 1.4 | 3.2 | 1.1 | 1.3 |
| 206177_s_at | ARG1 | 1.3E−04 | 1.2 | 3.1 | −1.1 | 2.7 |
| 203554_x_at | PTTG1 | 2.6E−14 | 2.0 | 3.0 | 1.8 | 1.8 |
| 206697_s_at | HP | 1.9E−04 | 1.5 | 2.9 | 1.1 | 2.1 |
| 209409_at | GRB10 | 1.4E−04 | 1.3 | 2.9 | −1.1 | 2.1 |
| 209138_x_at | IGL@ | 2.0E−08 | 1.7 | 2.9 | 1.4 | 1.7 |
| 217179_x_at | — | 1.7E−03 | 1.3 | 2.9 | −1.0 | 1.2 |
| 206641_at | TNFRSF17 | 5.4E−04 | 1.2 | 2.8 | 1.2 | 1.9 |
| 206513_at | AIM2 | 4.6E−06 | 2.4 | 2.8 | 1.5 | 2.0 |
| 206576_s_at | CEACAM1 | 2.4E−04 | 1.7 | 2.8 | 1.3 | 1.5 |
| 202589_at | TYMS | 4.8E−08 | 1.8 | 2.7 | 1.8 | 1.9 |
| 202870_s_at | CDC20 | 6.2E−11 | 1.6 | 2.7 | 1.6 | 1.7 |
| 221253_s_at | MUTED/TXNDC5 | 2.1E−05 | 1.4 | 2.7 | 1.3 | 1.8 |
| 216510_x_at | IGHA1/IGHG1/IGHM/IGHV3-23/IGHV4-31/ | 7.5E−04 | 1.2 | 2.7 | 1.1 | 1.3 |
| | LOC100290375 | | | | | |
| 202503_s_at | KIAA0101 | 3.0E−05 | 1.9 | 2.6 | 1.4 | 1.6 |
| 209369_at | ANXA3 | 3.2E−06 | 1.6 | 2.5 | 1.2 | 1.9 |
| 214677_x_at | CYAT1/IGLV1-44 | 6.6E−10 | 1.7 | 2.5 | 1.3 | 1.6 |
| 214669_x_at | IGKC | 1.2E−10 | 1.6 | 2.5 | 1.3 | 1.6 |
| 214916_x_at | IGH@/IGHA1/IGHA2/IGHG1/IGHG3/ | 6.0E−04 | 1.1 | 2.4 | 1.0 | 1.2 |
| | IGHM/IGHV3-23/IGHV4-31/ | | | | | |
| | LOC100290375 | | | | | |
| 211372_s_at | IL1R2 | 1.4E−04 | 1.1 | 2.4 | −1.1 | 2.2 |
| 209498_at | CEACAM1 | 2.5E−05 | 1.5 | 2.4 | 1.4 | 1.7 |
| 217227_x_at | IGLV1-44/LOC100290557 | 8.6E−04 | 1.2 | 2.4 | 1.1 | 1.3 |
| 206464_at | BMX | 1.1E−05 | 1.4 | 2.4 | 1.1 | 1.7 |
| 219978_s_at | NUSAP1 | 1.2E−10 | 1.6 | 2.4 | 1.6 | 1.6 |
| 205041_s_at | ORM1/ORM2 | 7.0E−08 | 1.0 | 2.4 | −1.1 | 1.4 |
| 211889_x_at | CEACAM1 | 1.2E−04 | 1.5 | 2.4 | 1.3 | 1.5 |
| 216491_x_at | IGHM | 4.1E−07 | 1.3 | 2.4 | 1.1 | 1.2 |
| 212531_at | LCN2 | 4.9E−06 | −1.2 | 2.4 | 1.2 | 1.9 |
| 216576_x_at | IGK@/IGKC/LOC652493/LOC652694 | 3.9E−05 | 1.4 | 2.3 | 1.2 | 1.4 |
| 221286_s_at | MGC29506 | 1.3E−03 | 1.2 | 2.3 | 1.1 | 1.4 |
| 211798_x_at | IGLJ3 | 4.9E−05 | 1.3 | 2.3 | 1.1 | 1.3 |
| 215379_x_at | IGLV1-44 | 5.4E−10 | 1.6 | 2.3 | 1.3 | 1.5 |
| 216984_x_at | IGLV2-23/LOC100293440 | 3.7E−05 | 1.3 | 2.3 | 1.1 | 1.6 |
| 217378_x_at | LOC100130100/LOC100291464 | 2.1E−06 | 1.4 | 2.3 | 1.2 | 1.4 |
| 201554_x_at | GYG1 | 2.2E−06 | 1.5 | 2.3 | 1.1 | 1.7 |
| 216207_x_at | IGKV1D-13 | 5.1E−06 | 1.5 | 2.3 | 1.2 | 1.5 |
| 217480_x_at | LOC100287723/LOC642424/LOC642838 | 1.3E−06 | 1.4 | 2.3 | 1.2 | 1.4 |
| 216560_x_at | IGL@ | 6.7E−04 | 1.3 | 2.3 | 1.2 | 1.3 |
| 202018_s_at | LTF | 1.2E−05 | −1.4 | 2.2 | 1.3 | 1.9 |
| 214836_x_at | IGK@/IGKC | 3.9E−08 | 1.4 | 2.2 | 1.3 | 1.4 |

TABLE 3

| Probe Set | Condition | 219669_at | 206576_s_at |
|---|---|---|---|
| Gene | Assessed | CD177 | CEACAM1 |
| Baseline | Threshold | 10 | 2 |
| Expression | True Positive | 9 | 10 |
| Across | True Negative | 111 | 98 |
| Population | False Positive | 2 | 15 |
| | False Negative | 40 | 39 |
| | Specificity | 0.98 | 0.87 |
| | Sensitivity | 0.18 | 0.20 |
| Baseline | Threshold | 10 | 2 |
| Expression | True Positive | 8 | 7 |
| from | True Negative | 113 | 105 |
| Individual | False Positive | 0 | 8 |
| Patient(s) | False Negative | 41 | 42 |
| | Specificity | 1.00 | 0.93 |
| | Sensitivity | 0.16 | 0.14 |

### Example 2

### Methods Used to Confirm Biomarkers for Predicting Gastrointestinal Immuned-Related Adverse Events in Patients Administered Costimulatory Pathway Inhibitors Using qPCR

The relationship between gene expression of the biomarkers disclosed herein and gastrointestinal immune-related adverse events was further evaluated using quantitative PCR.

### Methods

qPCR Analysis. A two-step qPCR assay was performed to confirm microarray profiling results for 15 of the top target genes. Eight hundred nanogram of total RNA was reverse transcribed into 20 μL of cDNA using the SUPERSCRIPT® Vilo cDNA synthesis kit (Invitrogen, Carlsbad, Calif.). The cDNA samples were mixed with ABI 2× TAQMAN® Universal PCR Master Mix and loaded into the TAQMAN® Low Density Array Card. A 384-well micro fluidiccard, pre-loaded with target genes and 2 housekeeping probe sets (ABI Assay-on-Demand TAQMAN® Gene Expression Assays, ABI, P/N 4342249) was run on ABI 7900HT Systems for relative quantitation according to manufacturer's instructions. The probe sets for the target genes analyzed are commercially available from ABI. Representative probe sets for CD177 and CEACAM1 were as follows: CD177 (Hs00360669_m1); and CEACAM1 (Hs00989783_m1). The expression data were normalized using ACT method with 18S as housekeeping gene.

Patient samples from the CA184-004 and CA184-007 trials were used for the qPCR analysis.

The same statistical method described for the gene expression in Example 1 herein was also applied to the qPCR data analysis as well.

The qPCR results are provided in Table 4. As shown, elevated CD177 expression was confirmed to be statistically correlated with the subsequent incidence of GI-irAE patients after administration of ipilimumab. The magnitude of this correlation significantly increased when the date the expression measurement was obtained post-administration of ipilimumab. The results for CEACAM1 show an increased correlation between elevated expression and incidence of GI-irAE relative to other genes measured. However, CEACAM1 did not meet statistical significance with this particular data set. In addition, most of the other genes measured also did not meet statistical significance with this particular data set. Additional patient samples from other ipilimumab trials will be analyzed.

TABLE 4

| Gene | P-val (GI) | P-val (Date) |
|---|---|---|
| CD177 | 4.91E−03 | 3.68E−14 |
| CEAMCAM1 | 9.55E−02 | 6.01E−02 |
| SLC45A2 | 1.05E−01 | 3.35E−02 |
| ELANE | 1.37E−01 | 6.30E−01 |
| SCUBE2 | 1.96E−01 | 5.20E−01 |
| IR2RG | 2.45E−01 | 2.95E−01 |
| CEACAM8 | 2.60E−01 | 4.68E−01 |
| OLFM4 | 2.61E−01 | 3.17E−01 |
| CD3E | 2.69E−01 | 4.69E−01 |
| LTF | 4.07E−01 | 6.32E−01 |
| RAB27A | 4.51E−01 | 7.27E−01 |
| RAB18 | 4.56E−01 | 1.84E−01 |
| IL32 | 4.90E−01 | 4.80E−02 |
| ID2 | 6.05E−01 | 6.28E−01 |
| SNCA | 6.49E−01 | 3.97E−02 |

### Example 3

### Methods Used to Confirm Biomarkers for Predicting Gastrointestinal Immuned-Related Adverse Events in Patients Administered Costimulatory Pathway Inhibitors in an Additional Patient Cohort

The relationship between gene expression of the biomarkers disclosed herein and gastrointestinal immune-related adverse events was further evaluated in an independent patient cohort.

Data from a phase I clinical trial (CA184078), a Randomized, Parallel, 3-arm Study to Characterize the Effect of Ipilimumab+Chemotherapy in Patients With Untreated Advanced Melanoma, was analyzed. (Vafa et al., J. Clin. Oncol., 30(Suppl.), Abstract 8593 (2012)) was used to confirm the relationship between gene expression of the biomarkers disclosed herein and gastrointestinal immune-related adverse events in the prior analyses. The methods utilized are outlined as follows.

### Methods

Whole blood samples from clinical study CA184078 were independently analyzed using the same statistical model as outlined in Example 1. In this study, 20 patients were treated with ipilimumab monotherapy at 10 mg/kg every 3 weeks for 4 doses. The mean CD177 expression ratio comparing GI-irAE and No-GI-irAE groups at week 3 and 11 was 4.3 and 12.0, respectively, with no significant difference at baseline. In the GI-irAE group, the mean fold change from baseline to week 3 and 11 was 4.8 and 15.3, respectively. By contrast, in the No-GI-irAE group, these changes were negligible (1.1 and 1.2, respectively). Expression changes similar to those seen in the other two studies were observed for CEACAM1 and most of the granule-associated genes, with significant changes from baseline to week 11 in the GI-irAE group but not in the No-GI-irAE group (see Table 5).

### Table 5

Granule-Associated Gene Expression Profiles in CA184078. (A) Mean expression ratio comparing the GI-irAE and No-GI-irAE groups for granule-associated genes at each time point. P value for the test of a difference in mean expression between the two GI-irAE groups (averaged over the three time points) also is shown. (B) Mean fold change from baseline (BL) in the GI-irAE and No-GI-irAE groups for granule-associated genes. P value for the test of a difference in mean expression among the three time points (averaged over the two groups) is shown. Mean expression ratio: positive values give (mean expression in the GI-irAE group)/(mean expression in the No-GI-irAE group); negative values give negative of (mean expression in the No-GI-irAE group)/(mean expression in the GI-irAE group). Mean fold change: positive values give mean of (post-baseline expression)/(baseline expression); negative values give negative mean of (baseline expression)/(post-baseline expression).

TABLE 5A
(CA184078)

| | | Mean expression ratio comparing | | | |
|---|---|---|---|---|---|
| | | GI-irAE and No-GI-irAE groups | | | |
| | | P | Base- | Week | Week |
| Probe Set | Gene | Value | line | 3 | 11 |
| 219669_at | CD177 | 4.4E−02 | −1.1 | 4.3 | 12.0 |
| 206576_s_at | CEACAM1 | 3.1E−04 | 1.1 | 1.9 | 2.8 |
| 209498_at | CEACAM1 | 5.8E−05 | 1.1 | 2.9 | 3.8 |
| 211889_x_at | CEACAM1 | 6.4E−06 | 1.1 | 1.8 | 2.7 |
| 214575_s_at | AZU1 | 2.3E−01 | 1.1 | −1.0 | 1.2 |
| 205557_at | BPI | 2.4E−03 | 1.6 | 1.6 | 3.6 |
| 210244_at | CAMP | 2.0E−03 | 1.4 | 1.5 | 4.0 |
| 203757_s_at | CEACAM6 | 3.5E−02 | 1.2 | 1.2 | 2.5 |
| 211657_at | CEACAM6 | 1.3E−01 | −1.2 | 1.0 | 1.8 |
| 206676_at | CEACAM8 | 5.3E−03 | 1.1 | 1.7 | 5.1 |
| 205653_at | CTSG | 7.4E−01 | 1.1 | 1.1 | 1.1 |
| 207269_at | DEFA4 | 1.5E−01 | 1.3 | 1.5 | 2.3 |
| 206871_at | ELANE | 1.9E−01 | 1.2 | 1.4 | 1.6 |
| 202018_s_at | LTF | 5.1E−03 | 2.2 | 1.7 | 4.7 |
| 203948_s_at | MPO | 6.6E−02 | 1.1 | 1.2 | 1.3 |
| 203949_at | MPO | 7.7E−02 | 1.1 | 1.1 | 1.3 |
| 212768_s_at | OLFM4 | 1.5E−02 | 1.1 | 1.6 | 7.8 |

TABLE 5B
(CA184078)

| | Mean fold change from | | Mean fold change from BL | |
|---|---|---|---|---|
| | BL in GI-irAE group | | in No-GI-irAE group | |
| P Value | BL vs. W3 | BL vs. W11 | BL vs. W3 | BL vs. W11 |
| 1.6E−01 | 4.8 | 15.3 | 1.1 | 1.2 |
| 8.7E−02 | 1.9 | 2.7 | 1.1 | 1.1 |
| 4.7E−02 | 2.3 | 3.6 | −1.2 | 1.0 |
| 1.0E−01 | 1.6 | 2.3 | 1.0 | −1.0 |
| 6.6E−01 | −1.1 | −1.0 | −1.0 | −1.1 |
| 1.8E−03 | −1.0 | 2.7 | −1.1 | 1.2 |
| 7.0E−04 | −1.1 | 2.7 | −1.2 | −1.0 |
| 1.8E−02 | −1.0 | 2.5 | −1.0 | 1.2 |
| 3.1E−02 | 1.1 | 1.9 | −1.1 | −1.1 |
| 8.1E−03 | 1.1 | 3.9 | −1.4 | −1.2 |
| 2.9E−01 | −1.3 | 1.0 | −1.3 | 1.0 |
| 6.9E−02 | −1.2 | 2.5 | −1.3 | 1.4 |
| 3.9E−01 | −1.2 | 1.4 | −1.4 | 1.0 |
| 1.6E−02 | −1.3 | 2.3 | −1.0 | 1.1 |
| 4.7E−01 | 1.1 | 1.2 | 1.0 | 1.1 |
| 3.7E−01 | 1.1 | 1.3 | 1.0 | 1.0 |
| 4.3E−03 | 1.3 | 7.2 | −1.0 | 1.1 |

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1. A method for predicting the likelihood a cancer patient may experience a gastrointestinal immune-related adverse event in response to therapy comprising the administration of a CTLA-4 antagonist, comprising the steps of:
(i) administering to said patient a therapy comprising a CTLA-4 antagonist;
(ii) measuring the expression level of CD177 in a sample from said patient collected subsequent to administration of said therapy;
(iii) comparing said measured expression level to a baseline or predetermined level of CD177 expression; and
(iv) classifying said patient as having an increased likelihood of experiencing a gastrointestinal immune-related adverse event in response to said therapy if said patient has an elevated level of CD177 expression relative to said baseline or predetermined level.

2. The method according to claim 1, wherein said elevated level of CD177 is observed within about 3 weeks subsequent to treatment.

3. The method according to claim 1, wherein said elevated level of CD177 is at least about 2 fold high than said baseline or predetermined level.

4. A method of treating an individual suffering from cancer with a therapy comprising the administration of a CTLA-4 antagonist comprising the steps of:
(i) administering to said patient a therapy comprising a CTLA-4 antagonist;
(ii) measuring the expression level of CD177 in a sample from said patient collected subsequent to administration of said therapy; and
(iii) comparing said measured expression level to a baseline or predetermined level of CD177 expression; and
(iv) introducing an alternative treatment regimen for said patient if an elevated level of CD177 expression relative to said baseline or predetermined level is observed.

5. The method according to claim 4, wherein said alternative treatment regimen comprises a regimen selected from the group consisting of:
(a) continuing to administer said therapy but altering said regimen by reducing the frequency of said administration;
(b) continuing to administer said therapy but altering said regimen by reducing the dose of said therapy;
(c) continuing to administer said therapy but altering said regimen by reducing both the dose or frequency of administration of said therapy;
(d) continuing to administer said therapy but interrupting the administration of said therapy for a period of time;
(e) continuing to administer said therapy but altering said regimen by introducing a steroid into the treatment regimen;
(f) continuing to administer said therapy but altering said regimen by introducing infliximab into the treatment regimen;
(g) continuing to administer said therapy but altering said regimen by introducing budesonide into the treatment regimen;
(h) continuing to administer said therapy but altering said regimen by introducing solumedrol into the treatment regimen;
(i) continuing to administer said therapy but altering said regimen by introducing a CD137 agonist into the treatment regimen; and
(j) continuing to administer said therapy but altering said regimen by introducing bowel rest and electrolytes into the treatment regimen.

6. The method according to claim 4, wherein if said elevated level of CD177 expression has an RMA of at least 8, recommending said therapy be discontinued for a period of time to either prevent or decrease the likelihood said patient may experience a gastrointestinal immune-related adverse event.

7. A method for predicting the likelihood a cancer patient may experience a gastrointestinal immune-related adverse event in response to therapy comprising the administration of a CTLA-4 antagonist, comprising the steps of:
(i) administering to said patient a therapy comprising a CTLA-4 antagonist;
(ii) measuring the expression level of CEACAM1 in a sample from said patient collected subsequent to administration of said therapy;
(iii) comparing said measured expression level to a baseline or predetermined level of CEACAM1 expression, and
(iv) classifying said patient as having an increased likelihood of experiencing a gastrointestinal immune-related adverse event in response to said therapy if said patient has an elevated level of CEACAM1 expression relative to said baseline or predetermined level.

8. The method according to claim 7, wherein said elevated level of CEACAM1 is observed within about 3 weeks subsequent to treatment.

9. The method according to claim 7, wherein said elevated level of CEACAM1 is at least about 2 fold high than said baseline or predetermined level.

10. A method of treating an individual suffering from cancer with a therapy comprising the administration of a CTLA-4 antagonist comprising the steps of:
(i) administering to said patient a therapy comprising a CTLA-4 antagonist;
(ii) measuring the expression level of CEACAM1 in a sample from said patient collected subsequent to administration of said therapy; and
(iii) comparing said measured expression level to a baseline or predetermined level of CEACAM1 expression; and
(iv) introducing an alternative treatment regimen for said patient if an elevated level of CEACAM1 expression relative to said baseline or predetermined level is observed.

11. The method according to claim 10, wherein said alternative treatment regimen comprises a regimen selected from the group consisting of:
(a) continuing to administer said therapy but altering said regimen by reducing the frequency of said administration;
(b) continuing to administer said therapy but altering said regimen by reducing the dose of said therapy;
(c) continuing to administer said therapy but altering said regimen by reducing both the dose or frequency of administration of said therapy;
(d) continuing to administer said therapy but interrupting the administration of said therapy for a period of time;
(e) continuing to administer said therapy but altering said regimen by introducing a steroid into the treatment regimen;
(f) continuing to administer said therapy but altering said regimen by introducing infliximab into the treatment regimen;
(g) continuing to administer said therapy but altering said regimen by introducing budesonide into the treatment regimen;
(h) continuing to administer said therapy but altering said regimen by introducing solumedrol into the treatment regimen;
(i) continuing to administer said therapy but altering said regimen by introducing a CD137 agonist into the treatment regimen; and
(j) continuing to administer said therapy but altering said regimen by introducing bowel rest and electrolytes into the treatment regimen.

12. The method according to claim 10, wherein if said elevated level of CEACAM1 expression has an RMA of at least 8, recommending said therapy be discontinued for a period of time to either prevent or decrease the likelihood said patient may experience a gastrointestinal immune-related adverse event.

13. The method of claim 1, wherein a recommended dose for said CTLA-4 antagonist is administered at a dosage of about 0.1 to 15 mg/kg once every three weeks.

14. The method of claim 1, wherein the CTLA-4 antagonist is selected from the group consisting of: ipilimumab and tremelimumab.

15. The method of claim 1, wherein said cancer is selected from the group consisting of: melanoma, prostate cancer, lung cancer, non-small cell lung cancer, small cell lung cancer, gastric cancer, ovarian cancer, brain cancer, and any other cancer disclosed herein.

16. A kit for use in determining a treatment regimen for an individual with cancer, comprising:
(i) a therapeutically effective dose of an anti-CTLA-4 antagonist;
(ii) a means for measuring CEACAM1 and/or CD-177 expression subsequent to the administration of said anti-CTLA-4 antagonist; and
(iii) instructions to continue to administer said anti-CTLA-4 antagonist if the level of said CEACAM1 and/or CD177 relative to a baseline or predetermined level is less than a threshold level, or to follow an alternative treatment regimen.

17. The method of claim 7, wherein said cancer is selected from the group consisting of: melanoma, prostate cancer, lung cancer, non-small cell lung cancer, small cell lung cancer, gastric cancer, ovarian cancer, brain cancer, and any other cancer disclosed herein.

18. The method of claim 7, wherein a recommended dose for said CTLA-4 antagonist is administered at a dosage of about 0.1 to 15 mg/kg once every three weeks.

19. The method of claim 7, wherein the CTLA-4 antagonist is selected from the consisting of: ipilimumab and tremelimumab.


SEQUENCE LISTING (4 sequences)

SEQ ID NO: 1 - protein, 108 aa, homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 2 - protein, 118 aa, homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 3 - protein, 9 aa, homo sapiens
IMDQVPFSV

SEQ ID NO: 4 - protein, 9 aa, homo sapiens
YLEPGPVTV